(19) United States
(12) Patent Application Publication
(10) Pub. No.: US 2026/0020769 A1
(43) Pub. Date: Jan. 22, 2026

(54) SYSTEM, METHOD, AND INFORMATION PROCESSING DEVICE

(71) Applicant: TERUMO KABUSHIKI KAISHA, Tokyo (JP)

(72) Inventors: Takayuki UCHIDA, Hiratsuka Kanagawa (JP); Tomonori HATTA, Hadano Kanagawa (JP)

(21) Appl. No.: 19/340,805

(22) Filed: Sep. 25, 2025

Related U.S. Application Data
(63) Continuation of application No. PCT/JP2024/008917, filed on Mar. 8, 2024.

(30) Foreign Application Priority Data
Mar. 28, 2023 (JP) 2023-052329

(51) Int. Cl. A61B 5/021; G06F 3/0482

(52) U.S. Cl. CPC A61B 5/021; G06F 3/0482

(57) ABSTRACT

A system for monitoring a cardiac condition of a patient includes a display, an interface connectable to a device configured to store measurement data including electrocardiogram data, cardiac sound data, and pulse wave data of the patient, a processor configured to execute the steps of acquiring the measurement data, determining whether there is a measurement abnormality in the data, upon determining that there is no measurement abnormality, determining a feature point of each of the electrocardiogram data, the cardiac sound data, and the pulse wave data, calculating time differences between two of the feature points, and determining whether there is a calculation abnormality, upon determining that there is no calculation abnormality, determining an intracardiac pressure based on the time differences, displaying information of the patient and the intracardiac pressure when determined.

### CROSS-REFERENCE TO RELATED APPLICATION

This application is a continuation of International Patent Application No. PCT/JP2024/008917 filed Mar. 8, 2024, which is based upon and claims the benefit of priority from Japanese Patent Application No. 2023-052329, filed Mar. 28, 2023, the entire contents of which are incorporated herein by reference.

### BACKGROUND

### Technical Field

The present disclosure relates to a system, a method, and an information processing device.

### Related Art

Patients with a history of cardiac failure are at risk of their cardiac functions gradually deteriorating even after their symptoms improve and they return to daily life. For such patients, it is known that the risk of onset of acute cardiac failure can be reduced by detecting signs of cardiac function deterioration at an early stage and providing appropriate treatment. Further, detecting cardiac function deterioration early in hospitalized patients and providing appropriate treatment is expected to decrease the in-hospital mortality rate. For that purpose, a pulse wave signal device has been proposed. This device measures pulse wave propagation time using an electrocardiogram, cardiac sound, and the pulse wave, and it also determines the quality of the measurement.

### SUMMARY

Pulse wave propagation time is an index suitable for indicating the state of arteriosclerosis, which is one of the causative diseases of cardiac failure. The causative diseases of cardiac failure include diseases other than arteriosclerosis, such as cardiomyopathy, myocardial infarction, and valvular heart disease. The proposed pulse wave signal device cannot detect deterioration in cardiac function due to a worsening causative disease other than arteriosclerosis.

One aspect aims to provide a system and the like for monitoring the state of the heart on a daily basis.

In one embodiment, a system for monitoring a cardiac condition of a patient, comprises: a display; an interface circuit connectable to a device configured to store measurement data including electrocardiogram data, cardiac sound data, and pulse wave data of the patient; a memory that stores a program; a processor configured to execute the program to perform the steps of: acquiring the measurement data from the device through the interface circuit, determining whether there is a measurement abnormality in the measurement data, upon determining that there is no measurement abnormality in the measurement data, determining a feature point of each of the electrocardiogram data, the cardiac sound data, and the pulse wave data, calculating time differences between any two of the feature points, and determining whether there is a calculation abnormality in the time differences, upon determining that there is no calculation abnormality, determining an intracardiac pressure based on the time differences, generating a first screen showing information of the patient and the intracardiac pressure when determined, and controlling the display to display the generated screen.

In one aspect, it is possible to provide a system and the like configured to monitor the state of the heart on a daily basis.

### BRIEF DESCRIPTION OF DRAWINGS

FIG. 1 is a diagram for explaining an outline of a monitoring system.

FIG. 2 is a diagram for explaining the configuration of the monitoring system.

FIG. 3 is a diagram for explaining the configuration of the monitoring system.

FIG. 4 is a diagram for explaining temporal index values.

FIG. 5 is a diagram for explaining a first model.

FIG. 6 is a diagram for explaining a second model.

FIG. 7 is a diagram for explaining the record layout in a patient database (DB).

FIG. 8 is a diagram for explaining the record layout in an analysis DB.

FIG. 9 is a flowchart of processing performed at a measurement stage.

FIG. 10 is a flowchart of processing performed at a data check stage.

FIG. 11 is a flowchart of processing for intracardiac pressure derivation.

FIG. 12 is a flowchart of processing for representative value calculation.

FIG. 13 is a flowchart of processing for intracardiac pressure change.

FIG. 14 is an example of a patient list screen.

FIG. 15 is an enlarged view of a portion XV in FIG. 14.

FIG. 16 is an enlarged view of a portion XVI in FIG. 14.

FIG. 17 is a diagram for explaining screen transition.

FIG. 18 is an example of a waveform screen.

FIG. 19 is an example of a waveform screen.

FIG. 20 is an example of a waveform screen.

FIG. 21 is an example of a transition screen.

FIG. 22 is an example of a transition screen.

FIG. 23 is an example of a detailed transition screen.

FIG. 24 is an example of a detailed transition screen.

FIG. 25 is a diagram for explaining another example of feature points.

FIG. 26 is a diagram for explaining a configuration of a second information processing device.

FIG. 27 is a diagram for explaining the record layout in a first training DB.

FIG. 28 is a flowchart of processing for generating the first model. and

FIG. 29 is a diagram for explaining a configuration of a monitoring system according to a third embodiment.

### DETAILED DESCRIPTION

### First Embodiment

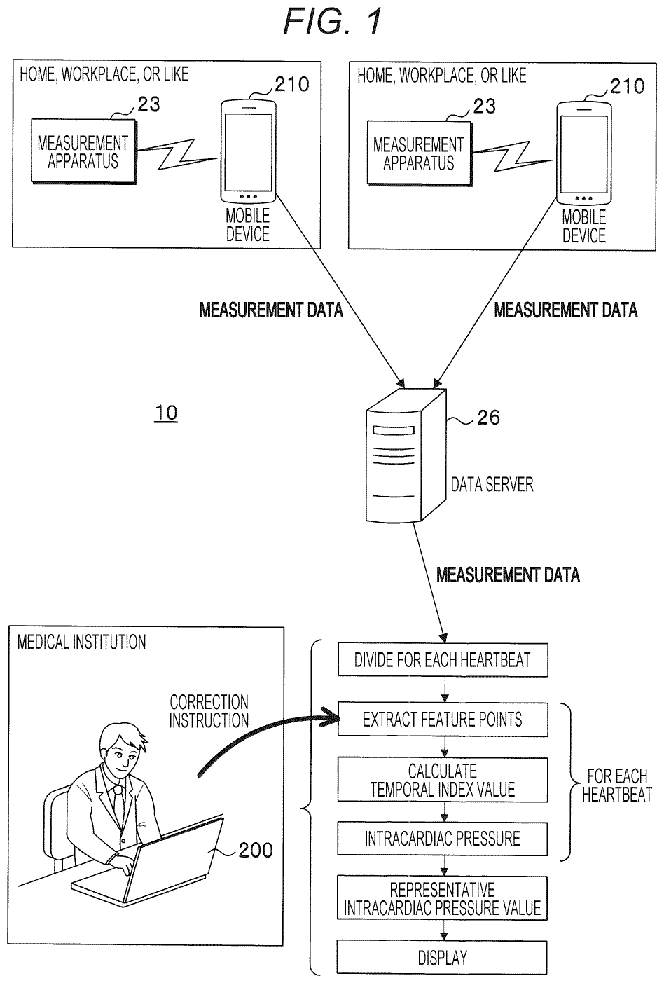

FIG. 1 is a diagram for explaining an outline of a monitoring system 10. The monitoring system 10 includes a measurement apparatus 23, a mobile device 210, an information processing device 200, and a data server 26. The monitoring system 10 is used for remote monitoring of a patient diagnosed with cardiac failure. The patient is a monitoring target, and measures the electrocardiogram, the cardiac sound, the pulse wave, and the like with the measurement apparatus 23 at home, a workplace, a nursing home, or a medical institution, for example. It is desirable to perform the measurement at least once daily. The measurement apparatus 23 will be described later in detail.

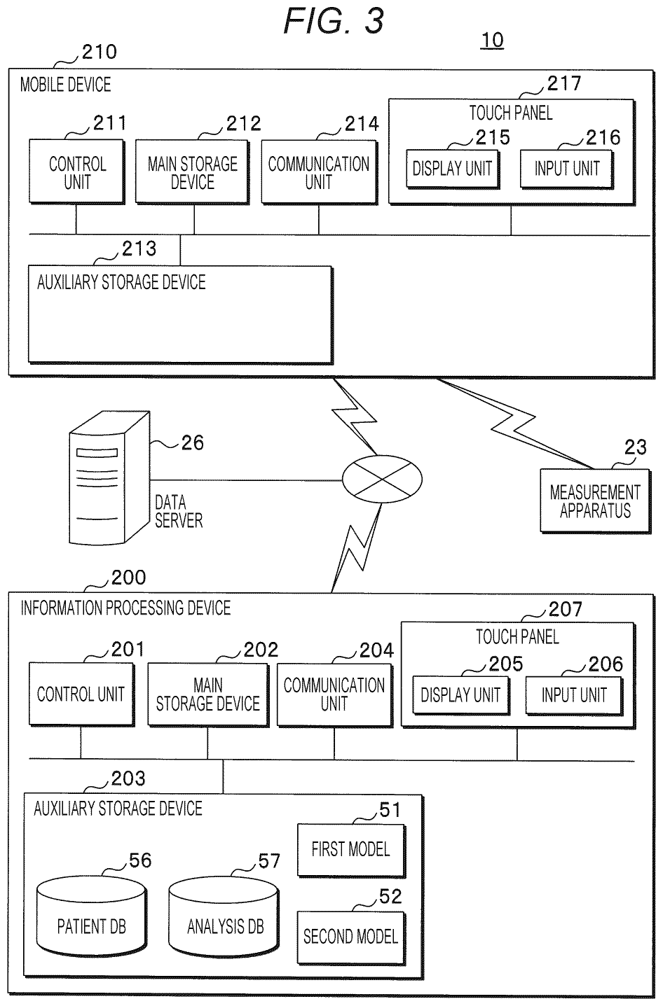

Measurement data is transmitted to the data server 26 via the mobile device 210 such as a smartphone. The doctor reads measurement data of a plurality of patients from the data server 26 into the information processing device 200. On the basis of the measurement data, the intracardiac pressures of the respective patients are derived, and are listed on a display unit 205 (see FIG. 3) of the information processing device 200. The method of deriving the intracardiac pressures and the list display will be described later in detail.

The intracardiac pressure is an index indicating the state of cardiac function. In cardiac failure, it is known that the intracardiac pressure increases before the onset of subjective symptoms. Therefore, by monitoring the intracardiac pressure, exacerbation of cardiac failure can be detected at an early stage. Cardiac failure is caused by myocardial infarction, angina pectoris, arteriosclerosis, hypertension, valvular disease, cardiomyopathy, arrhythmia, and the like. Examples of the intracardiac pressure include values or waveforms of right atrial pressure (systolic pressure, diastolic pressure, average pressure), right ventricular pressure (systolic pressure, diastolic pressure, end-diastolic pressure), pulmonary artery pressure (systolic pressure, diastolic pressure, average pressure), left atrial pressure (systolic pressure, diastolic pressure, average pressure), left ventricular pressure (systolic pressure, diastolic pressure, end-diastolic pressure), and femoral arterial pressure.

In a case where a patient suspected of exacerbation of cardiac failure is found by checking of the data displayed as the list, the doctor recommends a visit to the hospital, or gives instruction for improvement of lifestyle, or the like. Instead of the doctor, a paramedical staff member under the instruction of the doctor may check the data, and ask the doctor for judgment as necessary. Measurement data of each patient is accumulated in the data server 26, and measurement data of a plurality of patients can be collectively checked at the doctor's convenience or the like, so that the burden on the doctor or the like can be reduced.

The onset of acute cardiac failure can be prevented by appropriate treatment before the onset of subjective symptoms. Since the patient can receive contact from the doctor before feeling subjective symptoms, the patient can live his/her daily life with greater peace of mind. Thus, quality of life (QOL) of the patient can be improved with the use of the monitoring system 10. Note that the outline of the data processing procedure shown in the lower right in FIG. 1 will be described later.

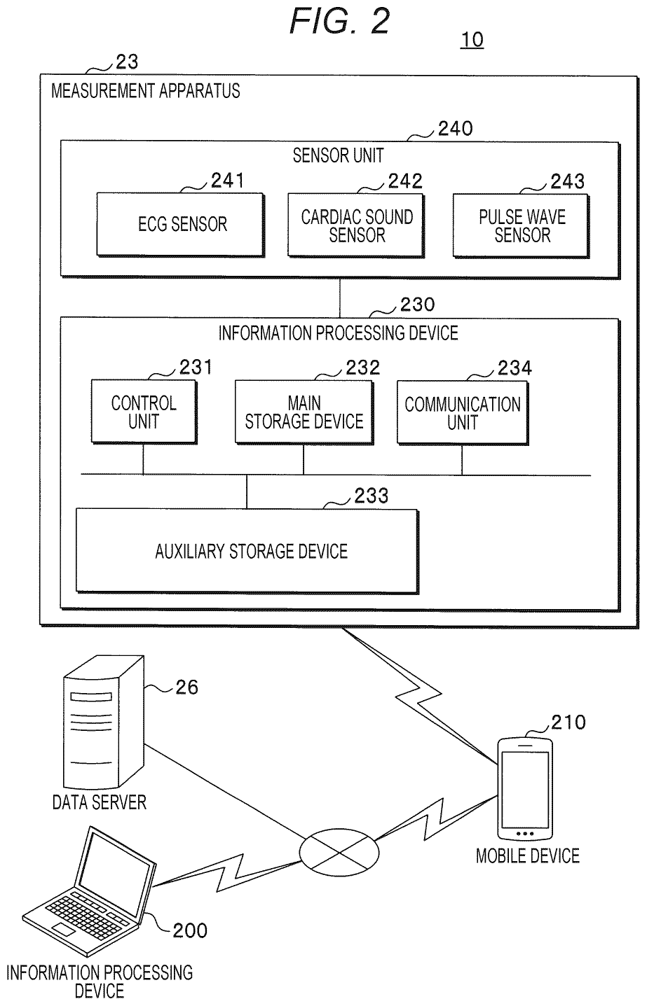

FIGS. 2 and 3 are diagrams for explaining the configuration of the monitoring system 10. The configuration of the measurement apparatus 23 is now described with reference to FIG. 2. The measurement apparatus 23 includes a sensor unit 240 and an information processing device 230. The sensor unit 240 includes an electrocardiogram (ECG) sensor 241, a cardiac sound sensor 242, and a pulse wave sensor 243.

In the sensor unit 240, the ECG sensor 241, the cardiac sound sensor 242, and the pulse wave sensor 243 are accommodated in one housing, for example, and the patient can simultaneously measure the electrocardiogram, the cardiac sound, and the pulse wave by disposing or fixing them in the vicinity of the heart by himself/herself. The sensor unit 240 does not have a housing, and the patient may place or attach the respective sensors in or to pre-specified positions. Since the ECG sensor 241, the cardiac sound sensor 242, and the pulse wave sensor 243 are conventionally used, detailed explanation of them is not made herein.

The information processing device 230 includes a control unit 231, a main storage device 232, an auxiliary storage device 233, a communication unit 234, and a bus. The control unit 231 is an arithmetic control device that executes a program according to the present embodiment. As the control unit 231, one or more processors, such as a central processing unit (CPU), a graphics processing unit (GPU), or a multi-core CPU, is used. Through the bus, the control unit 231 is connected to each of the hardware components constituting the information processing device 230.

The main storage device 232 is a storage device such as a static random access memory (SRAM), a dynamic random access memory (DRAM), or a flash memory. The main storage device 232 temporarily stores information necessary during the process to be performed by the control unit 231, and the program being executed by the control unit 231.

The auxiliary storage device 233 is a storage device such as an SRAM, a flash memory, a hard disk, or a magnetic tape. The auxiliary storage device 233 stores a program to be executed by the control unit 231, and various kinds of data necessary for executing the program. The communication unit 234 is a network interface circuit that conducts communication between the measurement apparatus 23 and the mobile device 210.

The information processing device 230 is a single board computer or a one-chip microcomputer, and is formed integrally with the sensor unit 240. The information processing device 230 may also be formed integrally with the ECG sensor 241, the cardiac sound sensor 242, or the pulse wave sensor 243. The information processing device 230 may be dedicated hardware for the measurement apparatus 23.

The measurement apparatus 23 may be formed integrally with a health management device such as a sphygmomanometer or a weight scale. The measurement apparatus 23 may be formed integrally with a so-called smartwatch worn by a patient on a daily basis.

Referring now to FIG. 3, the configurations of the mobile device 210 and the information processing device 200 are described. The mobile device 210 includes a control unit 211, a main storage device 212, an auxiliary storage device 213, a communication unit 214, a touch panel 217, and a bus. The touch panel 217 includes a display unit 215 and an input unit 216.

The control unit 211 is an arithmetic control device that executes a program according to the present embodiment. As the control unit 211, one or more processors, such as a CPU, a GPU, or a multi-core CPU, is used. Through the bus, the control unit 211 is connected to each of the hardware components constituting the mobile device 210.

The main storage device 212 is a storage device such as an SRAM, a DRAM, or a flash memory. The main storage device 212 temporarily stores information necessary during the process to be performed by the control unit 211, and the program being executed by the control unit 211. The auxiliary storage device 213 is a storage device such as an SRAM, a flash memory, a hard disk, or a magnetic tape. The auxiliary storage device 213 stores a program to be executed by the control unit 211, and various kinds of data necessary for executing the program.

The communication unit 214 is a network interface circuit that conducts communication between the mobile device 210 and the measurement apparatus 23, and communication between the mobile device 210 and the network. For example, near field communication (NFC) such as Bluetooth (registered trademark) is used for communication between the mobile device 210 and the measurement apparatus 23. A commercial communication line, a local area network (LAN), or the like is used for communication between the mobile device 210 and the network.

The display unit 215 is a liquid crystal display (LCD) panel or an organic electro-luminescence (EL) panel, for example. The input unit 216 is stacked on the display unit 215. The mobile device 210 may include the input unit 216 such as a keyboard, a mouse, a voice input microphone, or a gesture input sensor, instead of the touch panel 217, or in addition to the touch panel 217. The mobile device 210 is an information processing device such as a smartphone, tablet, or personal computer.

The information processing device 200 includes a control unit 201, a main storage device 202, an auxiliary storage device 203, a communication unit 204, a touch panel 207, and a bus. The touch panel 207 includes a display unit 205 and an input unit 206.

The control unit 201 is an arithmetic control device that executes a program according to the present embodiment. As the control unit 201, one or more processors, such as a CPU, a GPU, or a multi-core CPU, is used. Through the bus, the control unit 201 is connected to each of the hardware components constituting the information processing device 200.

The main storage device 202 is a storage device such as an SRAM, a DRAM, or a flash memory. The main storage device 202 temporarily stores information necessary during the process to be performed by the control unit 201, and the program being executed by the control unit 201.

The auxiliary storage device 203 is a storage device such as an SRAM, a flash memory, a hard disk, or a magnetic tape. The auxiliary storage device 203 stores a first computer model 51 (hereinafter simply referred to as the first model 51), a second computer model 52 (hereinafter simply referred to as the first model 52), a patient data base (DB) 56, an analysis DB 57, the program to be executed by the control unit 201, and various kinds of data necessary for executing the program. The patient DB 56 and the analysis DB 57 may be stored in an external large-capacity storage device connected to the information processing device 200.

The communication unit 204 is a network interface circuit that conducts communication between the information processing device 200 and the network. The display unit 205 is an LCD panel or an organic EL panel, for example. The input unit 206 is stacked on the display unit 205. The information processing device 200 may include the input unit 206 such as a keyboard, a mouse, a voice input microphone, or a gesture input sensor, instead of the touch panel 207, or in addition to the touch panel 207.

The information processing device 200 is a personal computer, a tablet, a large computing machine, a virtual machine that runs in a large computing machine, or a quantum computer. The information processing device 200 may be formed with a plurality of personal computers that perform distributed processing, or hardware such as a large computing machine. The information processing device 200 may be formed with a cloud computing system. The information processing device 200 may be formed with a plurality of personal computers that operate simultaneously, or hardware such as a large computing machine.

The data server 26 is a large-capacity storage device connected to the network. The data server 26 records data transmitted from the information processing device 200 and the mobile device 210, and transmits data requested from the information processing device 200 and the mobile device 210. The patient DB 56 and the analysis DB 57 are stored not only in the auxiliary storage device 203 but also in the data server 26, and data may be synchronized by a replication process. For example, it is possible to provide the monitoring system 10 that can operate the information processing device 200 disposed in each of the examination rooms and the nurse station in the same manner. The data server 26 may be realized by a cloud service.

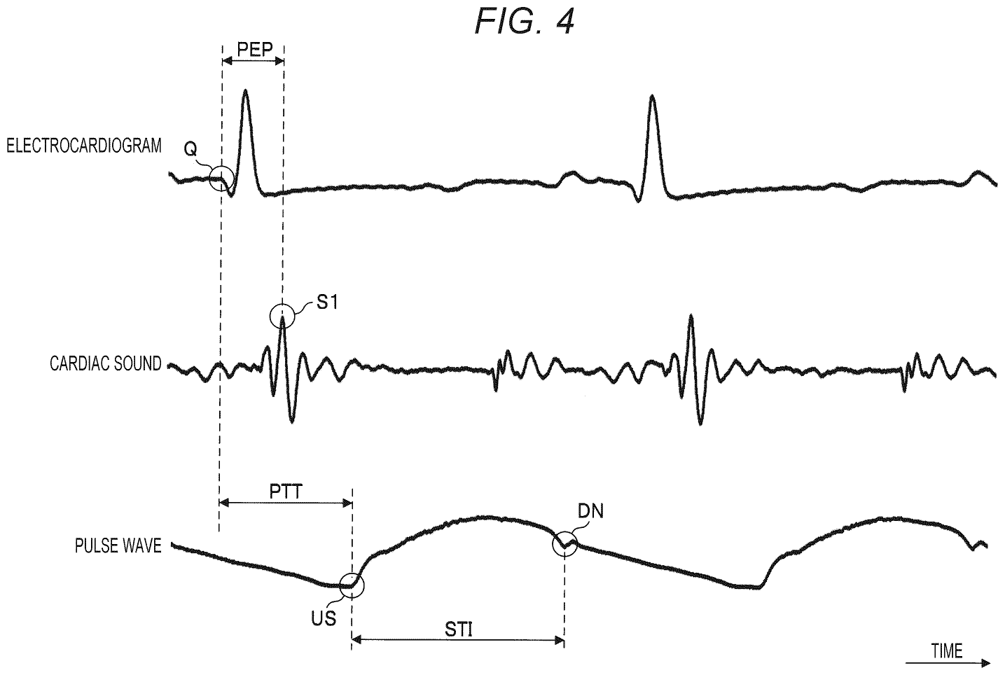

FIG. 4 is a diagram for explaining temporal index values. Waveforms indicating electrocardiogram data, cardiac sound data, and pulse wave data are shown in this order from the top. These three waveforms indicate data measured simultaneously from one patient. The horizontal direction in FIG. 4 indicates the passage of time, and the data closer to the right end indicates newer data.

The vertical axis of the electrocardiogram indicates the voltage measured by the ECG sensor 241, the vertical axis of the cardiac sound indicates the sound pressure of the cardiac sound measured by the cardiac sound sensor 242, and the vertical axis of the pulse wave indicates the vascular pressure measured by the pulse wave sensor 243. FIG. 4 shows waveforms of about two heartbeats, but in one measurement, waveforms of several tens of heartbeats are measured. A temporal index value is an index indicating a temporal relationship among the electrocardiogram data, the cardiac sound data, and the pulse wave data, or a temporal change in any one piece of the electrocardiogram data, the cardiac sound data, and the pulse wave data.

Feature points of the respective waveforms of the electrocardiogram data, the cardiac sound data, and the pulse wave data to be used in the present embodiment are now described. Regarding the electrocardiogram, the start position of a Q wave is a feature point Q. Regarding the cardiac sound, the position indicating a highest sound pressure is a feature point S1. Regarding the pulse wave, the position at which the blood flow rate exhibits a minimum value is a feature point US (Up-Stroke) indicating an ejection start point, and the position at which a small valley-like notch is shown while the blood flow rate is dropping is a feature point DN (Dicrotic Notch). Since any of the feature points is commonly used in the field of cardiovascular medicine, a detailed definition thereof is not explained herein.

The temporal index values to be used in the present embodiment are now described. An index PEP (Pre-Ejection Period) is the elapsed time from the feature point Q to the feature point S1. An index PTT (Pulse Transition Time) is the elapsed time from the feature point Q to the feature point US. An index STI (Systolic Time) is the elapsed time from the feature point US to the feature point DN. A temporal index value is an index indicating a temporal relationship (time difference) between feature points in at least two pieces of data among the electrocardiogram data, the cardiac sound data, and the pulse wave data, or a temporal relationship (time difference) between feature points in any one piece of the electrocardiogram data, the cardiac sound data, and the pulse wave data. Since any of the indexes is commonly used in the field of cardiovascular medicine, the medical meaning thereof is not explained herein.

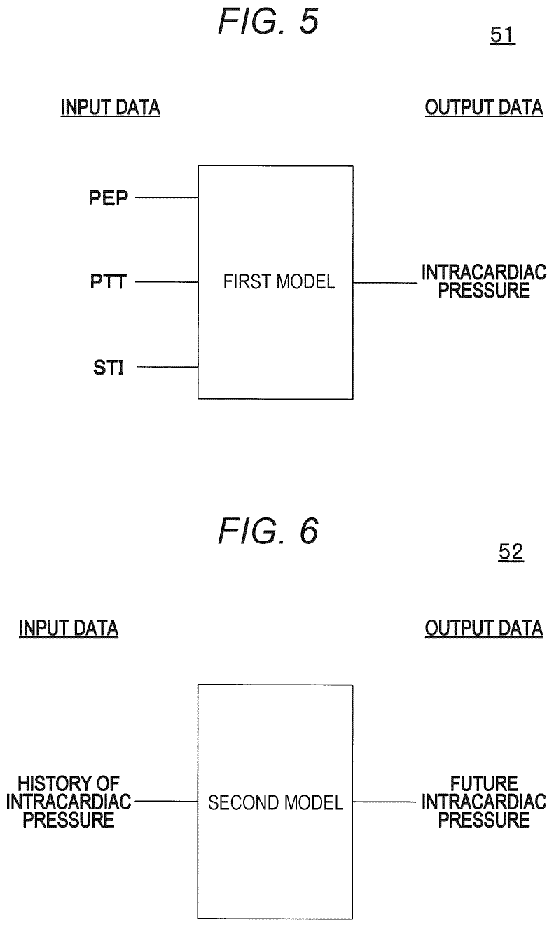

FIG. 5 is a diagram for explaining the first model 51. The first model 51 receives inputs of the index PEP, the index PTT, and the index STI, and outputs an estimated value of the intracardiac pressure of the patient. The intracardiac pressure to be output includes, but is not limited to, a left heart pressure, a right heart pressure, a left ventricular pressure waveform, a right ventricular pressure waveform, a left ventricular end-diastolic pressure (LVEDP), a left atrium pressure (LAP), a left ventricular pressure (LVP), a pulmonary artery end-diastolic pressure (PAEDP) or a pulmonary artery diastolic pressure (PADP), a right atrium pressure (RAP), a right ventricular pressure (RVP), a central venous pressure (CVP), a pulmonary artery pressure (PAP), and a pulmonary wedge pressure (PWP), for example. The pulmonary wedge pressure is also called a pulmonary arterial wedge pressure (PAWP), a pulmonary capillary wedge pressure (PCWP), or a pulmonary artery occlusion pressure (PAOP). Since the intracardiac pressure varies periodically every heartbeat, the intracardiac pressure includes a systolic pressure, a diastolic pressure, and an average pressure.

The first model 51 outputs values obtained by estimating representative values of the intracardiac pressure in one heartbeat, for example. The representative values are the diastolic intracardiac pressure, which is the minimum value of the intracardiac pressure in one heartbeat, the systolic intracardiac pressure, which is the maximum value of the intracardiac pressure in one heartbeat, and the average value of the intracardiac pressure in one heartbeat. A first model 51 that outputs the value obtained by estimating the diastolic intracardiac pressure, a first model 51 that outputs the value obtained by estimating the systolic intracardiac pressure, and a first model 51 that outputs the value obtained by estimating the average value of the intracardiac pressure may be prepared. The first model 51 may output the waveform obtained by estimating the pressure waveform of the intracardiac pressure.

In the description below, unless otherwise specified, the intracardiac pressure will be described using the systolic intracardiac pressure as an example. Further, in the description below, a case where the intracardiac pressure is the pulmonary artery pressure will be described as an example. That is, in the description below, a case where the intracardiac pressure to be output by the first model 51 is an estimated value of the systolic pulmonary artery pressure will be described as an example. “PAP” in example screens and the like described later is an acronym for the English term “pulmonary artery pressure”, which means pulmonary artery pressure. However, the intracardiac pressure to be calculated and displayed by the monitoring system 10 is not limited to the systolic pulmonary artery pressure.

The first model 51 is a trained model that has been trained to receive inputs of the index PEP, the index PTT, and the index STI in one heartbeat, and output an estimated value of the intracardiac pressure of the patient in the same heartbeat, for example. The training method in a case where the first model 51 is a trained model will be described later. The first model 51 is an example of an intracardiac pressure model that outputs an intracardiac pressure when temporal index values are input.

The first model 51 may be a function that derives an estimated value of the intracardiac pressure, using the index PEP, the index PTT, and the index STI as arguments. The function for deriving the intracardiac pressure can be created by analyzing the index PEP, the index PTT, the index STI, and actual measured data of the intracardiac pressure, using the least-square method, for example.

Referring back to FIG. 1, the description is continued. As described above, several tens of heartbeats are measured in one measurement, and measurement data is stored into the data server 26. The control unit 201 divides the measurement data for each heartbeat, for example. The control unit 201 extracts feature points of the electrocardiogram data, the cardiac sound data, and the pulse wave data of each divided heartbeat as described with reference to FIG. 4, and calculates a temporal index value (elapsed time) from a temporal relationship (time difference, time interval) between two feature points in one heartbeat.

The two feature points may be two feature points included in one piece of data (two feature points in one piece of the electrocardiogram data, two feature points in one piece of the cardiac sound data, or two feature points in one piece of the pulse wave data, for example), or may be two feature points in each piece of data of two or more pieces of data (one feature point in the electrocardiogram data and one feature point in the cardiac sound data, one feature point in the electrocardiogram data and one feature point in the pulse wave data, or one feature point in the cardiac sound data and one feature point in the pulse wave data). Note that the control unit 201 may divide the measurement data for each adjacent heartbeat interval. An adjacent heartbeat interval is a time interval between feature points of adjacent heartbeats, such as an RR interval that is the interval from one QRS wave in the electrocardiogram to another QRS wave that appears next timewise, for example. The control unit 201 may calculate a temporal index value (elapsed time) from a temporal relationship (time difference) between two feature points in an adjacent heartbeat interval.

The control unit 201 inputs the calculated temporal index values to the first model 51, and acquires the intracardiac pressure for each heartbeat. The control unit 201 determines a representative value of the acquired intracardiac pressure for each of several tens of heartbeats. The control unit 201 outputs the representative values to the touch panel 207.

In the above manner, it is possible to provide the monitoring system 10 that is hardly affected by fluctuations in each one heartbeat and measurement errors.

For example, the feature point S1 can be simply defined as the maximum value of the cardiac sound in one heartbeat. However, from a medical point of view, the feature point S1 corresponds to a sound generated at the timing when the mitral valve and the tricuspid valve close, and is not the maximum value of the cardiac sound in some cases. The doctor checks the data as necessary, and corrects the feature points and the like extracted by the control unit 201. On the basis of the correction instruction, the control unit 201 again derives and displays the intracardiac pressure.

FIG. 6 is a diagram for explaining the second model 52. The second model 52 receives a history of intracardiac pressure (for example, the intracardiac pressure data of a predetermined period such as the past week or the past month) as input data, and outputs a prediction of future intracardiac pressure as output data. For the prediction, accumulated data of intracardiac pressure transitions is used as training data for machine learning or the like, for example, and future change in the intracardiac pressure is predicted from the past intracardiac pressure history, which will be described later in greater detail. The prediction of future intracardiac pressure is a prediction of time-series change in the intracardiac pressure within a predetermined period of time, such as one month or one year. The prediction of future intracardiac pressure may be a prediction of the maximum value of the intracardiac pressure within a predetermined period of time. The prediction of future intracardiac pressure may be a prediction of the intracardiac pressure at a predetermined time, such as one week or one month later.

As the second model 52, a time-series analysis algorithm such as Autoregressive Integrated Moving Average (ARIMA) or Prophet can be used, for example. The second model 52 may be generated by supervised machine learning using a long short-term memory (LSTM) network, for example.

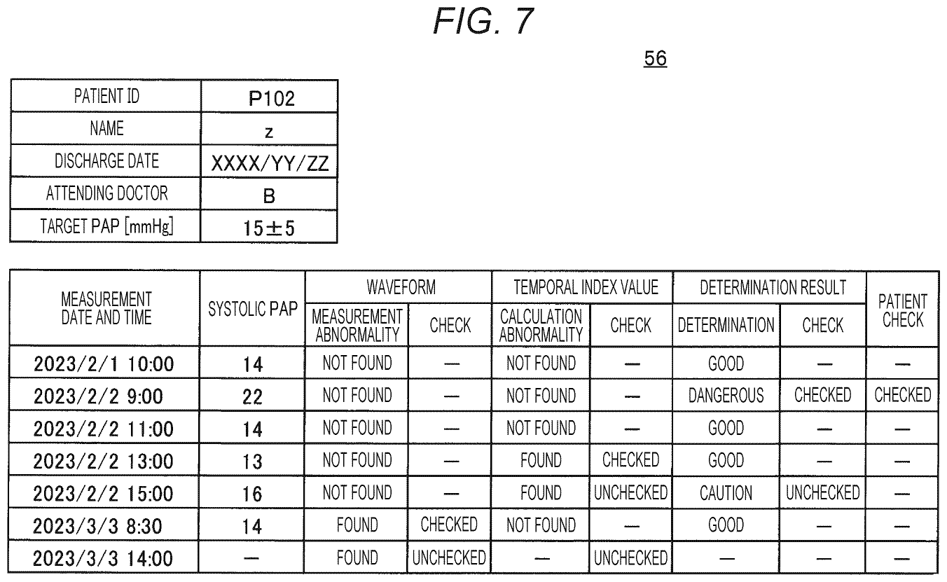

FIG. 7 is a diagram for explaining the record layout in the patient DB 56. The patient DB 56 is a DB that records basic information regarding the patient such as the patient identifier (ID) and the patient's name, the intracardiac pressure, and the original data used for estimation of the intracardiac pressure, which are associated with one another.

The patient DB 56 includes a patient ID field, a name field, a discharge date field, an attending doctor field, a target PAP field, a measurement date and time field, a systolic PAP field, a waveform field, a temporal index value field, a determination result field, and a patient check field. The waveform field includes a measurement abnormality field and a check field. The temporal index value field includes a calculation abnormality field and a check field. The determination result field includes a determination field and a check field.

In the patient ID field, a patient ID uniquely assigned to the patient is recorded. In the name field, the name of the patient is recorded. In the discharge date field, the discharge date determined when the patient was first hospitalized with symptoms of cardiac failure is recorded. In the attending doctor field, the attending doctor of the patient is recorded.

In the target PAP field, the median and the width of the management target value of the pulmonary artery pressure that is set by the attending doctor for each patient are recorded. In the present embodiment, the management target value is determined by the systolic pulmonary artery pressure. The name of the patient, the discharge date, the attending doctor, and the target PAP are acquired from an electronic medical record system (not shown) or the like with the patient ID being used as the key, and are recorded into the patient DB 56.

In the measurement date and time field, the dates and times at which measurement data such as the electrocardiogram was acquired with the measurement apparatus 23 are recorded. In the systolic PAP field, representative values of the systolic pulmonary artery pressure obtained on the basis of the measurement data are recorded. In the systolic PAP field, “-” indicates that any representative value of the systolic pulmonary artery pressure is not set.

In the measurement abnormality field, whether there is an abnormality in the measurement data of the electrocardiogram, the cardiac sound, and the pulse wave is recorded. In the check field, whether the abnormality in the measurement data has been checked by the doctor or the like is recorded. Here, “-” indicates that the measurement data does not have any abnormality, and therefore, does not need to be checked. “Checked” indicates that the abnormality in the measurement data has been checked. “Unchecked” indicates that the measurement data needs to be checked because of an abnormality, but has not been checked.

In the calculation abnormality field, whether there is an abnormality in a result of calculation of the temporal index values is recorded. In the check field, whether the calculation result has been checked by the doctor or the like is recorded. Here, “-” indicates that the calculation result does not have any abnormality, and therefore, does not need to be checked. “Checked” indicates that the abnormality in the calculation result has been checked. “Unchecked” indicates that the calculation result needs to be checked because of an abnormality, but has not been checked.

In the determination field, a result of determination as to the value of the pulmonary artery pressure is recorded. “Good” indicates that the systolic pulmonary artery pressure is equal to or lower than the target value recorded in the target PAP field, and a prediction that the systolic pulmonary artery pressure will not exceed the target value in the future has been output from the second model 52. “Dangerous” means that the systolic pulmonary artery pressure exceeds the target value. “Caution” indicates that the systolic pulmonary artery pressure is equal to or lower than the target value, but a prediction that the systolic pulmonary artery pressure will exceed the target value in the future has been output from the second model 52.

In the check field, whether the patient's state has been checked by the doctor is recorded. Here, “-” indicates that the determination result is “good”, and therefore, checking is not necessary. “Checked” indicates that the patient's state has been checked. “Unchecked” indicates the patient's state has not been checked.

In the patient check field, whether the doctor or the paramedical staff who has received an instruction from the doctor has contacted the patient and checked the health condition or the like is recorded. The check is made by telephone or the like. The patient may be asked to visit the hospital, and the doctor may conduct medical examination. Here, “-” indicates that checking has not been performed. “Checked” indicates that checking has been performed. The checked contents are recorded into an electronic medical record or the like. The patient DB 56 has one record for one data measurement.

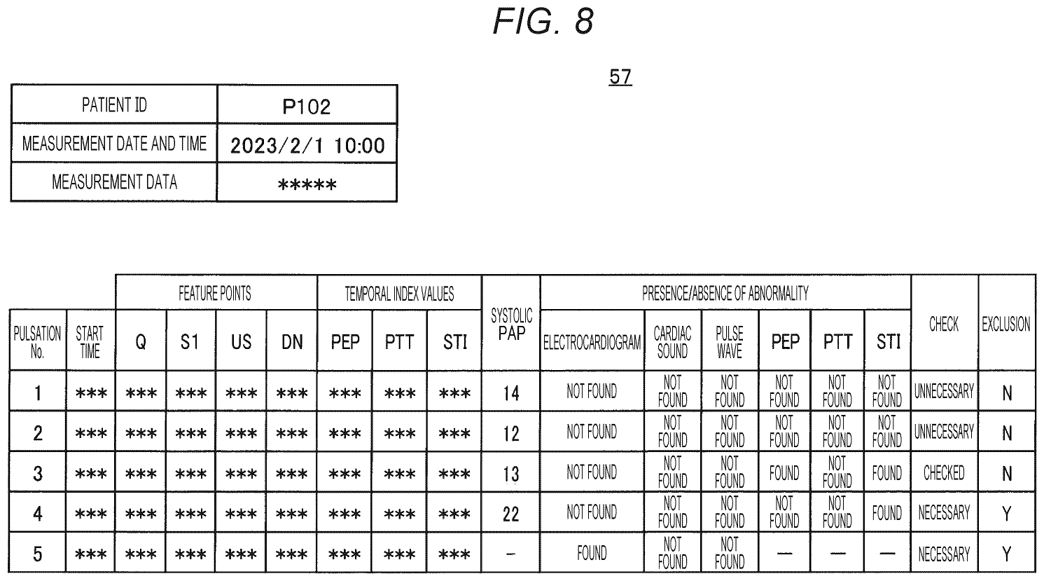

FIG. 8 is a diagram for explaining the record layout in the analysis DB 57. The analysis DB 57 is a DB that records the position at which the measurement data is divided for each heartbeat, the feature points, the temporal index values, the pulmonary artery pressure, and the like of each heartbeat, and the presence or absence of abnormality in the waveforms and the temporal index values, which are associated with one another. In FIG. 8, each “***” represents a numerical value.

The analysis DB 57 includes a patient ID field, a measurement date and time field, a measurement data field, a heartbeat number field, a start time field, a feature point field, a temporal index value field, a systolic PAP field, an abnormality presence/absence field, a check field, and an exclusion field. The feature point field includes a Q field, an S1 field, a US field, and a DN field. The temporal index value field includes a PEP field, a PTT field, and an STI field. The abnormality presence/absence field includes an electrocardiogram field, a cardiac sound field, a pulse wave field, a PEP field, a PTT field, and an STI field.

In the patient ID field, a patient ID uniquely assigned to the patient is recorded. In the measurement date and time field, the dates and times at which the patient performed measurement with the measurement apparatus 23 are recorded. In the measurement data field, measurement data of electrocardiogram data, cardiac sound data, and pulse wave data obtained by one measurement is recorded in the comma separated values (CSV) format, for example. In the measurement data field, information necessary for reading the measurement data, such as the file name of the file in which the measurement data is recorded, may be recorded.

In the heartbeat number field, numbers obtained by dividing measurement data for each heartbeat and assigning serial numbers to the divided data are recorded. In the start time field, the start time of each heartbeat is recorded. Note that the end time of each heartbeat is the same as the time recorded in the start time field of the record with the next heartbeat number.

In each subfield in the feature point field, the respective times corresponding to the feature point Q, the feature point S1, the feature point US, and the feature point DN described with reference to FIG. 4 are recorded. In each subfield in the temporal index value field, the respective temporal index values calculated on the basis of the times recorded in the feature point field are recorded. In the systolic PAP field, the systolic pulmonary artery pressures output from the first model 51, to which temporal index values were input, are recorded.

In each of the electrocardiogram field, the cardiac sound field, and the pulse wave field, whether there is a measurement abnormality in the waveform of each one heartbeat is recorded. Here, a measurement abnormality in the waveform means an abnormality caused by a measurement state such as a state in which a sensor is not disposed or fixed at an appropriate position, a state in which a sensor is broken, or a state in which accidental external noise caused by body movement, respiration, or the like has appeared, for example.

For example, the control unit 201 performs pattern matching with a waveform sample of a case where various abnormalities occur in measurement, determines that there is an abnormality when the similarity is high, and records the abnormality in the analysis DB 57. In a case where there is a measurement abnormality in any of the waveforms, the feature points and the pulmonary artery pressure cannot be appropriately determined. Therefore, as shown in the data of heartbeat No. 5, “-” is recorded in the feature point field, the temporal index value field, and the PAP field.

In the PEP field, the PTT field, and the STI field in the abnormality presence/absence field, presence/absence of abnormality in the respective temporal index values is recorded. Here, “-” means that any temporal index value has not been calculated, and therefore, presence or absence of abnormality in the temporal index value is not determined. Whether there is an abnormality in a temporal index value is determined on the basis of a difference from the average value of the temporal index values calculated from data of several tens of heartbeats obtained by one measurement, for example. Specifically, in a case where a temporal index value is out of the range of the average value ±3 σ, the control unit 201 determines that there is an abnormality. Here, a means standard deviation.

In the check field, whether the doctor needs to check each heartbeat is recorded. In a case where “found” is recorded in any of the subfields in the abnormality presence/absence field, the control unit 201 determines that checking by the doctor is required, and records “necessary” in the check field. “Checked” indicates that checking has been performed by the doctor. In a case where “not found” is recorded in any of the subfields of the abnormality presence/absence field, the control unit 201 determines that checking by the doctor is unnecessary, and records “unnecessary” in the check field.

In the exclusion field, whether the heartbeat is to be excluded in a case where the representative value of the pulmonary artery pressure acquired for each heartbeat is calculated is recorded. “N” means that the heartbeat is not to be excluded, and “Y” means that the heartbeat is to be excluded. For example, the control unit 201 records “Y” in the exclusion field of each record having “necessary” recorded in the check field, and excludes the record from the representative value calculation. In a case where the doctor checks the data and determines that the data should not be excluded, the control unit 201 changes the data in the exclusion field from “Y” to “N”, and again performs the representative value calculation. The analysis DB 57 includes one record for each one heartbeat.

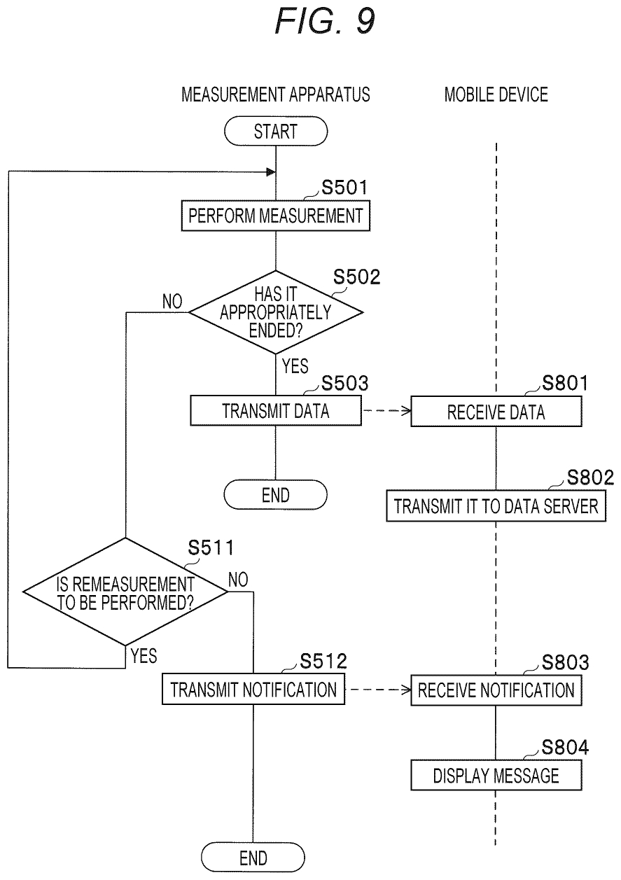

FIG. 9 is a flowchart of processing performed according to a program at a measurement stage. The program in FIG. 9 is started after the patient places or fixes the sensor unit 240 onto his/her body. The control unit 231 measures the electrocardiogram, the cardiac sound, and the pulse wave via the sensor unit 240 (step S501). The control unit 231 determines whether the measurement has appropriately ended (step S502). For example, in a case where the sensor unit 240 is detached from the body during the measurement, in a case where the remaining capacity of the built-in battery is insufficient, in a case where a large amount of noise is generated, in a case where normal measurement data is not obtained for a predetermined number of beats (ten beats, for example) or more, or in a case where a predetermined proportion (90%, for example) or more of normal measurement data is not obtained, the control unit 231 determines that the measurement has not appropriately ended. Note that, as for a medical measurement device such as an electrocardiograph, the function of determining whether measurement has appropriately ended is known, and therefore, a detailed explanation of the determination method is not made herein.

If it is determined that the measurement has appropriately ended (YES in step S502), the control unit 231 transmits the measurement data to the mobile device 210 (step S503). After that, the control unit 231 ends the processing. The control unit 211 receives the measurement data (step S801). The control unit 211 adds information such as the patient ID and the measurement date and time to the measurement data, and transmits the measurement data to the data server 26 (step S802). The data server 26 stores the received information.

If it is determined that the measurement data has not appropriately ended (NO in step S502), the control unit 231 determines whether to conduct remeasurement of measurement data (electrocardiogram data, cardiac sound data, and pulse wave data, for example) (step S511). In a case where the measurement has been repeated a predetermined number of times or more, for example, the control unit 231 determines not to conduct remeasurement. If it is determined that remeasurement is to be conducted (YES in step S511), the control unit 231 returns to step S501.

If it is determined that remeasurement is not to be conducted (NO in step S511), the control unit 231 transmits a notification indicating that the measurement has not been appropriately performed to the mobile device 210 (step S512). After that, the control unit 231 ends the processing.

The control unit 211 receives the notification (step S803). The control unit 211 displays, on the display unit 215, a message notifying the patient that the measurement could not be performed, such as “Measurement failed. Attach the sensor unit correctly and try again” (step S804). The control unit 211 may display, on the display unit 215, a video or the like explaining the method of correctly attaching the sensor unit 240.

Note that the control unit 231 may determine whether measurement has been appropriately performed for each heartbeat, and, if the measurement has not been appropriately performed, transmit a notification to the mobile device 210. It is possible to provide the measurement apparatus 23 that promptly notifies the patient in a case where the sensor unit 240 is not correctly attached or the like.

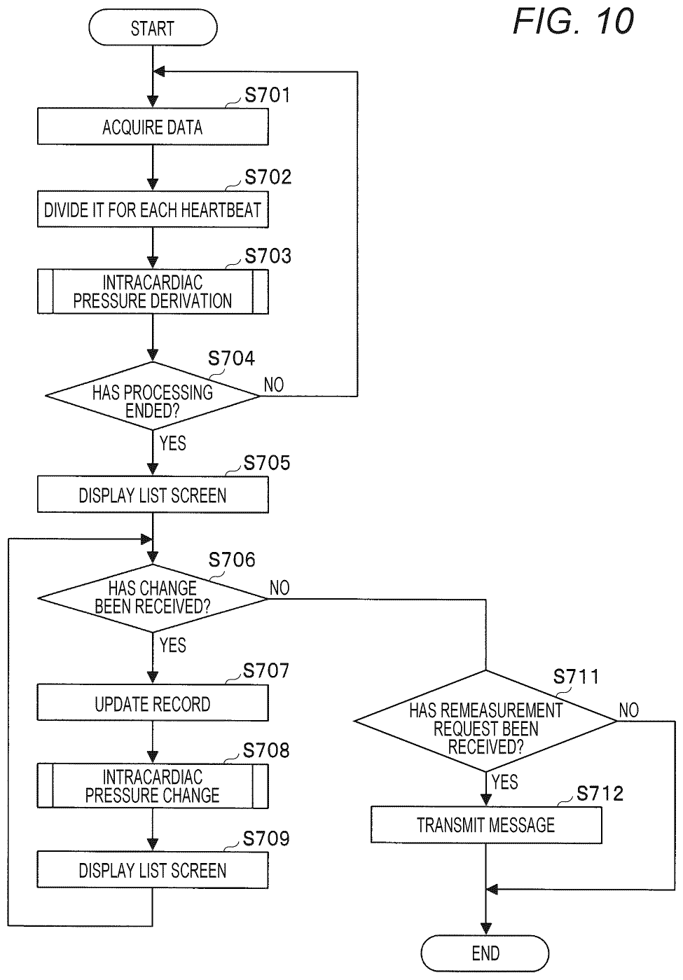

FIG. 10 is a flowchart of processing performed according to the program at the data check stage. The program in FIG. 10 is used when a medical care professional such as a doctor checks data of a plurality of patients and determines the presence or absence of signs of cardiac function deterioration.

The control unit 201 acquires one set of unprocessed measurement data from the data server 26 (step S701). The control unit 201 creates a new record in the analysis DB 57, and records the measurement data acquired from the data server 26 in the measurement data field and the measurement date and time in the measurement date and time field.

The control unit 201 divides the measurement data for each heartbeat (step S702). For example, the control unit 201 analyzes the electrocardiogram and determines a start point of the P wave to be a heartbeat separator. The start point of the P wave indicates the time when activation of the right atrium started. The control unit 201 may set a time at which the interval between the R waves indicating the maximum amplitude is divided at a predetermined ratio, as a heartbeat separator. Automatic analysis of electrocardiogram has been conventionally performed, and therefore, detailed explanation thereof is not made herein.

The control unit 201 records the serial number in the heartbeat number field in the analysis DB 57, and records the start time of the heartbeat in the start time field. The control unit 201 creates and records a record in the analysis DB 57 for all the heartbeats obtained by dividing the measurement data.

The control unit 201 invokes a subroutine for intracardiac pressure derivation (step S703). The subroutine for intracardiac pressure derivation is a subroutine for deriving the intracardiac pressure for each heartbeat included in the measurement data, calculating a representative value of the intracardiac pressure from the data of one measurement, and recording each piece of data in the analysis DB 57. The flow of processing according to the subroutine for intracardiac pressure derivation will be described later.

The control unit 201 determines whether the measurement data processing has ended (step S704). If it is determined that the processing has not ended (NO in step S704), the control unit 201 returns to step S701. If it is determined that the processing has ended (YES in step S704), the control unit 201 displays, on the touch panel 207, a patient list screen 71 (see FIG. 14) showing states of a plurality of patients on one screen (step S705). A specific example of the patient list screen 71 will be described later.

The control unit 201 determines whether an instruction to change a position of a feature point, a heartbeat separator position, or the like has been received via the touch panel 207 (step S706). When it is determined that an instruction to change has been received (YES in step S706), the control unit 201 updates the data recorded in the analysis DB 57 on the basis of the contents of the received instruction (step S707). For example, in a case where an instruction to change the feature point S1 in the cardiac sound waveform is received, the control unit 201 searches the analysis DB 57 to extract a record related to the heartbeat for which the instruction to change has been received. The control unit 201 updates the data recorded in the S1 field.

The control unit 201 invokes a subroutine for intracardiac pressure change (step S708). The subroutine for intracardiac pressure change is a subroutine for recalculating a representative value of the intracardiac pressure after deriving the intracardiac pressure for the heart rate for which the instruction to change has been received. The flow of processing according to the subroutine for intracardiac pressure change will be described later. The control unit 201 displays the patient list screen 71 using the updated data on the touch panel 207 (step S709). The control unit 201 then returns to step S706.

If it is determined that an instruction to change has not been received (NO in step S706), the control unit 201 determines whether an instruction to request remeasurement from the patient has been received (step S711). In a case where the doctor determines that remeasurement should be performed without waiting for the next day, such as a case where there is a possibility that correct measurement has not been performed, the doctor issues an instruction to request remeasurement.

If it is determined that an instruction to request remeasurement has been received (YES in step S711), the control unit 201 transmits a message, such as “Please remeasure today's data.”, to the mobile device 210 being used by the subject patient by short message service (SMS), e-mail, or the like (step S712). If it is determined that an instruction to request remeasurement has not been received (NO in step S711), or after the end of step S712, the control unit 201 ends the processing.

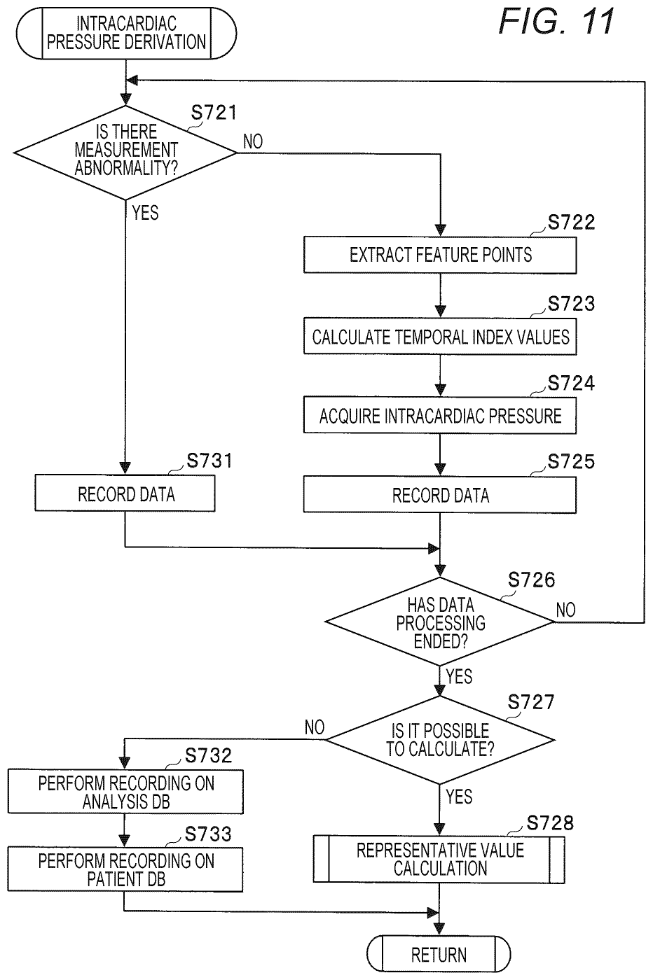

FIG. 11 is a flowchart of processing performed according to the subroutine for intracardiac pressure derivation. The subroutine for intracardiac pressure derivation is a subroutine for deriving the intracardiac pressure for each heartbeat included in the measurement data, calculating a representative value of the intracardiac pressure from the data of one measurement, and recording each piece of data in the analysis DB 57.

The control unit 201 determines whether there is a measurement abnormality in the waveforms of the electrocardiogram, the cardiac sound, and the pulse wave of one heartbeat (step S721). A measurement abnormality in a waveform means an abnormality caused by a measurement state. The control unit 201 performs pattern matching between a measured waveform and the respective abnormal waveform samples measured when various abnormalities occurred in the measurement, for example, and determines that there is an abnormality when the measured waveform is similar to one of the abnormal waveform samples.

If it is determined that there is not an abnormality in any of the waveforms of the electrocardiogram, the cardiac sound, and the pulse wave (NO in step S721), the control unit 201 extracts a feature point Q that is a feature point of the waveform of the electrocardiogram, a feature point S1 that is a feature point of the waveform of the cardiac sound, a feature point US that is a feature point of the waveform of the pulse wave, and a feature point DN (step S722).

On the basis of the feature points, the control unit 201 calculates the index PEP, the index PTT, and the index STI, which are temporal index values (step S723). The control unit 201 inputs the index PEP, the index PTT, and the index STI to the first model 51, and acquires the intracardiac pressure output from the first model 51 (step S724).

The control unit 201 creates a new record in the analysis DB 57, and records data (step S725). Specifically, the control unit 201 records the serial number in the heartbeat number field, and records the start time of the heartbeat being processed in the start time field. The control unit 201 records the times corresponding to the feature points extracted in step S722 in the respective subfields of the feature point field. The control unit 201 records the temporal index values calculated in step S723 in the respective subfields of the temporal index value field.

The control unit 201 records the intracardiac pressure acquired in step S724 in the systolic PAP field. The control unit 201 records “not found” in the electrocardiogram field, the cardiac sound field, and the pulse wave field in the abnormality presence/absence field.

If it is determined that there is a measurement abnormality (YES in step S721), the control unit 201 creates a new record in the analysis DB 57, and records data (step S731). Specifically, the control unit 201 records the serial number in the heartbeat number field, and records the start time of the heartbeat being processed in the start time field. The control unit 201 records “-” in each subfield of the feature point field and the temporal index value field, and in the PAP field.

The control unit 201 records “found” in the field corresponding to the waveform determined to have a measurement abnormality in step S721 among the electrocardiogram field, the cardiac sound field, and the pulse wave field, and records “not found” in the other fields.

Through the above processing, at the end of step S725 or step S731, the PEP field, the PTT field, and the STI field of the abnormality presence/absence field, the check field, and the exclusion field are in an undetermined state.

After the end of step S725 or step S731, the control unit 201 determines whether the processing of the data acquired in one measurement has ended (step S726). If it is determined that the processing has not ended (NO in step S726), the control unit 201 returns to step S721.

If it is determined that the processing has ended (YES in step S726), the control unit 201 determines whether a representative value of the intracardiac pressure can be calculated (step S727). For example, in a case where the number or proportion of the heartbeats determined to have a measurement abnormality in step S721 is equal to or smaller than a predetermined threshold, the control unit 201 determines that a representative value can be calculated. Through step S727, it is possible to prevent calculation of a representative value of the intracardiac pressure with low reliability, in a case where fixation of the sensor unit 240 is insufficient and a measurement abnormality frequently occurs, for example.

If it is determined that the calculation is possible (YES in step S727), the control unit 201 invokes a subroutine for representative value calculation (step S728). The subroutine for calculating a representative value is a subroutine for calculating a representative value of the intracardiac pressure on the basis of the data acquired in one measurement, and recording the calculated representative value in the DB. The flow of processing according to the subroutine for representative value calculation will be described later.

If it is determined that the calculation is not possible (NO in step S727), the control unit 201 records, in the analysis DB 57, information to the effect that a representative value of the intracardiac pressure cannot be calculated (step S732). Specifically, the control unit 201 searches the analysis DB 57 using the patient ID and the measurement date and time as keys, and extracts the records corresponding to the data of one measurement.

The control unit 201 records “-” in the PEP fields, the PTT fields, and the STI fields of all the extracted records. The control unit 201 records “necessary” in the check fields and “Y” in the exclusion fields of all the extracted records.

The control unit 201 creates a new record corresponding to the patient being processed in the patient DB 56, and records the date and time at which the data was measured in the measurement date and time field, and “-” in the patient check field. The control unit 201 records, in the new record, information to the effect that a representative value of the intracardiac pressure cannot be calculated (step S733). Specifically, the control unit 201 records “-” in the PAP field.

The control unit 201 searches the analysis DB 57 using the patient ID and the measurement date and time as keys, and extracts the records corresponding to the data of one measurement. The recording in the waveform field in the patient DB 56 is now described. In a case where “found” is not recorded in any of the electrocardiogram fields, the cardiac sound fields, and the pulse wave fields of the abnormality presence/absence fields of the records extracted from the analysis DB 57, the control unit 201 records “not found” in the measurement abnormality field and “-” in the check field in the patient DB 56. Otherwise, the control unit 201 records “found” in the measurement abnormality field, and records “unchecked” or “checked” in the check field.

Note that the control unit 201 may record “found” in the measurement abnormality field in the patient DB 56, in a case where the number of records in which “found” is recorded in the electrocardiogram field, the cardiac sound field, and the pulse wave field of the abnormality presence/absence field exceeds a predetermined threshold among the records extracted from the analysis DB 57.

Since the control unit 201 has not calculated the temporal index values, “-” is recorded in the calculation abnormality field of the temporal index value field, and “not yet” is recorded in the check field, in a case where it is not to be determined whether the temporal index values have an abnormality. The control unit 201 records “-” in each subfield of the determination result field and the patient check field. After the end of step S728 or step S733, the control unit 201 ends the processing.

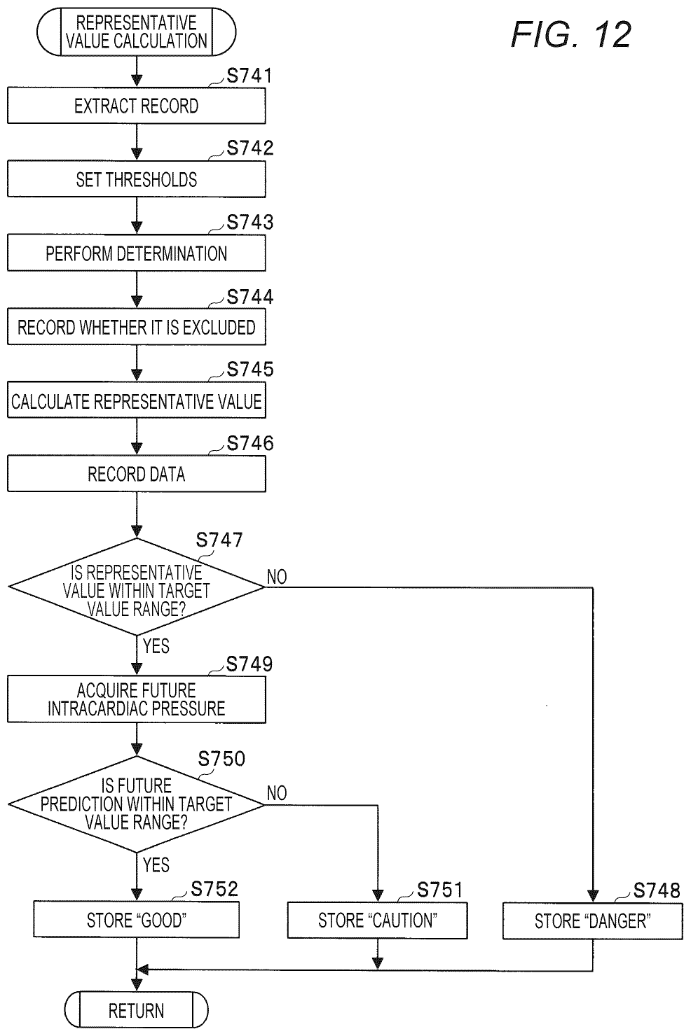

FIG. 12 is a flowchart of processing performed according to the subroutine for representative value calculation. The control unit 201 determines whether the temporal index values recorded in the temporal index value field in the analysis DB 57 include an outlier significantly different from the temporal index values of the other heartbeats in the same data. In the description below, a case where each temporal index value that is out of the range of average value ±3 σ is determined to be an outlier will be specifically described as an example.

The control unit 201 searches the analysis DB 57 using the patient ID and the measurement date and time as keys, and extracts the records corresponding to the data of one measurement (step S741). The control unit 201 calculates average values and standard deviations of the values recorded in the PEP fields, the PTT fields, and the STI fields. The control unit 201 sets the average value−3 σ and the average value+3 σ as thresholds for each temporal index value (step S742).

Note that the method of determining the thresholds in step S742 is not limited to the average value ±3 σ. For example, the average value ±σ or the average value ±2 σ may be set as the thresholds. Instead of setting thresholds in step S742 and determining whether it is within the range of the thresholds in step S743, it may be determined whether each temporal index value is so-called outlier data, using a statistical technique such as Smirnov-Grubbs test or Tietjen-Moore test, or a clustering technique such as X-means.

The control unit 201 determines whether the values recorded in the PEP field, the PTT field, and the STI field of the temporal index value field of each record fall within the range of the thresholds determined in step S742, and records the determination result (step S743). Specifically, the control unit 201 records “found” in the corresponding field of the abnormality presence/absence field in a case where the value is out of the range of the thresholds, and records “not found” in the corresponding field of the abnormality presence/absence field in a case where the value is within the range of the thresholds or is equal to the average value ±3 σ. Note that, in a case where “-” is recorded in the PEP field, the PTT field, or the STI field of the temporal index value field, the control unit 201 also records “-” in the corresponding fields of the abnormality presence/absence field.

The control unit 201 records “necessary” in the check field in a case where “found” or “-” is recorded in any of the subfields of the abnormality presence/absence field, and records “unnecessary” in the check field in a case where “not found” is recorded in all the subfields.

For each of the records extracted in step S741, the control unit 201 determines and records whether to exclude the measurement abnormality from the subject for which a representative value of the intracardiac pressure is to be calculated (step S744). Specifically, in a case where “found” is recorded in any of the subfields of the abnormality presence/absence field, the control unit 201 determines to exclude the data determined to have a measurement abnormality from the subject for representative value calculation, and records “Y” in the exclusion field. In a case where “not found” is recorded in all the subfields of the abnormality presence/absence field, the control unit 201 determines not to exclude the data from the subject for representative value calculation, and records “N” in the exclusion field.

The control unit 201 calculates a representative value of the intracardiac pressure (step S745). Specifically, the control unit 201 extracts the records in which “N” is recorded in the exclusion fields, from among the records extracted in step S741. The control unit 201 calculates a representative value of the intracardiac pressures recorded in the PAP fields of the extracted records. The representative value is an arithmetic average, for example. The representative value may be a median, a geometric mean, a harmonic mean, or the like.

The control unit 201 records the data into the patient DB 56 (step S746). Specifically, the control unit 201 searches the patient DB 56 using the patient ID and the measurement date and time as keys, and extracts the records corresponding to the data of one measurement. Note that, in a case where any record is not extracted, the control unit 201 creates a new record corresponding to the patient being processed in the patient DB 56, and records the date and time at which the data was measured in the measurement date and time field, and “-” in the patient check field. The control unit 201 records the representative value calculated in step S745 in the PAP field.

In a case where the records extracted from the analysis DB 57 in step S741 include a record in which “found” is recorded in any of the electrocardiogram field, the cardiac sound field, and the pulse wave field, the control unit 201 records “found” in the measurement abnormality field in the record being processed in the patient DB 56, and records “unchecked” in the check field. In a case where “found” is not recorded in any of the electrocardiogram field, the cardiac sound field, and the pulse wave field, the control unit 201 records “not found” in the measurement abnormality field and “-” in the check field.

In a case where the records extracted from the analysis DB 57 in step S741 include a record in which “found” or “-” is recorded in any of the PEP field, the PTT field, and the STI field of the abnormality presence/absence field, the control unit 201 records “found” in the calculation abnormality field in the record being processed in the patient DB 56, and records “unchecked” in the check field. In a case where “not found” is recorded in all the PEP field, PTT field, and the STI field, the control unit 201 records “not found” in the calculation abnormality field and “-” in the check field.

The control unit 201 determines whether the representative value of the intracardiac pressure calculated in step S745 is within the range of the target value recorded in the target PAP field (step S747). If it is determined that the representative value is out of the range of the target value (NO in step S747), the control unit 201 stores “danger” in the determination field of the determination result field of the record being processed and “unchecked” in the check field (step S748). After that, the control unit 201 ends the processing.

If it is determined that the representative value is within the range of the target value (YES in step S747), the control unit 201 extracts the PAP history of the patient being processed from the PAP field of the patient DB 56, inputs the history to the second model 52, and acquires the future intracardiac pressure output from the second model 52 (step S749). The control unit 201 determines whether the future intracardiac pressure acquired in step S748 is within the range of the target value (step S750).

If it is determined that the future intracardiac pressure is out of the range of the target value (NO in step S750), the control unit 201 stores “caution” in the determination field of the determination result field of the record being processed and “unchecked” in the check field (step S751). After that, the control unit 201 ends the processing.

If it is determined that the future intracardiac pressure is within the range of the target value (YES in step S750), the control unit 201 stores “good” in the determination field of the determination result field of the record being processed and “unchecked” in the check field (step S752). After that, the control unit 201 ends the processing.

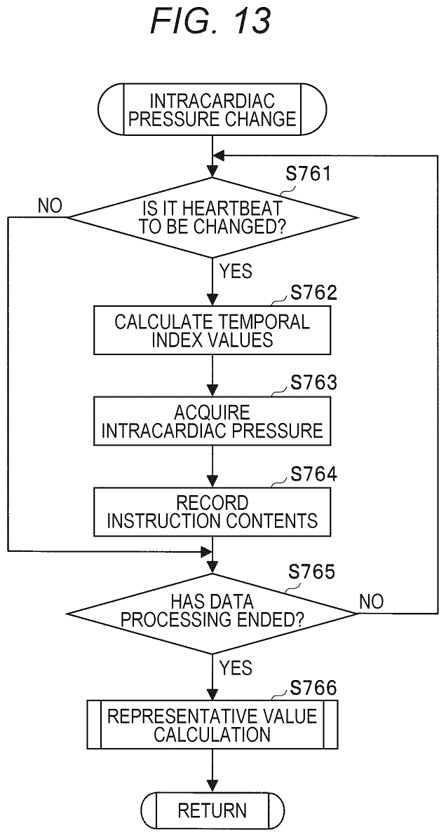

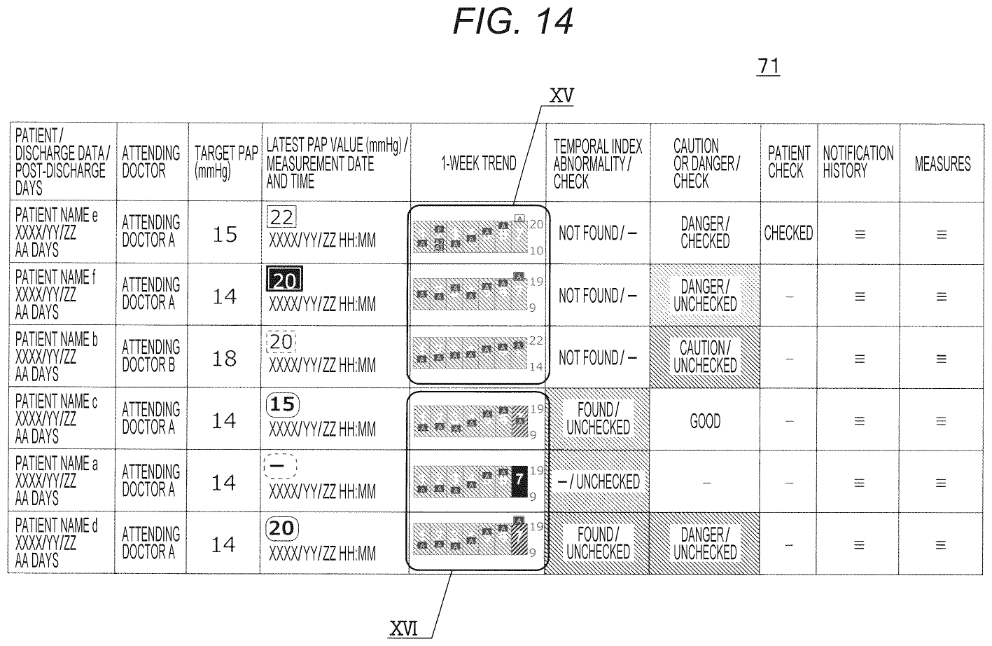

FIG. 13 is a flowchart of processing performed according to the subroutine for intracardiac pressure change. The subroutine for intracardiac pressure change is a subroutine for recalculating a representative value of the intracardiac pressure after deriving the intracardiac pressure for the heart rate for which the instruction to change has been received. In the subroutine for intracardiac pressure change, the control unit 201 sequentially processes the processing target data recorded in the analysis DB 57, starting from the first heartbeat.

The control unit 201 determines whether the heartbeat being processed is a heartbeat for which an instruction to change the positions of the feature points of the electrocardiogram data, the cardiac sound data, and the pulse wave data, the position of a heartbeat separator, or the like from the user has been received (step S761). If it is determined that an instruction to change has been received for the heartbeat (YES in step S761), the control unit 201 calculates the temporal index values on the basis of the changed feature points (step S762). The control unit 201 inputs the temporal index values to the first model 51, and acquires the intracardiac pressure output from the first model 51 (step S763).

The control unit 201 extracts records from the analysis DB 57, using the patient ID, the measurement date and time, and the heartbeat number as keys. The control unit 201 records the feature points designated by the user, the temporal index values calculated in step S762, and the intracardiac pressure acquired in step S763, in the feature point field, the temporal index value field, and the PAP field, respectively. The control unit 201 records “checked” in the check field. The control unit 201 records the contents of the instruction from the user in the exclusion field (step S764).

If it is determined that an instruction to change has not been received for the heartbeat (NO in step S761) or after the end of step S764, the control unit 201 determines whether the processing of the data acquired in one measurement has ended (step S765). If it is determined that the processing has not ended (NO in step S765), the control unit 201 returns to step S761.

If it is determined that the processing has ended (YES in step S765), the control unit 201 invokes a subroutine for representative value calculation (step S766). The subroutine for calculating a representative value is a subroutine for calculating a representative value of the intracardiac pressure on the basis of the data acquired in one measurement, and recording the calculated representative value in the DB. The flow of processing according to the subroutine for representative value calculation is the same as the flow of processing according to the subroutine described with reference to FIG. 12. After that, the control unit 201 ends the processing.

FIG. 14 is an example of the patient list screen 71. Information regarding a plurality of patients is listed in a table. A “patient/discharge date/post-discharge days” column shows patient names, and the discharge dates and post-discharge days at the time when the patient was first hospitalized due to a seizure such as acute cardiac failure. The post-discharge days are automatically calculated on the basis of the discharge date.

An “attending doctor” column shows the names of the attending doctors of the patients. A “target PAP” column shows the medians of the target values of the pulmonary artery pressure. The information in the above three columns is displayed on the basis of the name field, the discharge date field, the attending doctor field, and the target PAP field in the patient DB 56.

A “latest PAP value (mmHg)/measurement date and time” column shows the latest intracardiac pressure recorded in the PAP field of the latest record in the patient DB 56 described with reference to FIG. 7. The indication mode (output mode) of the intracardiac pressure is linked to the contents written in the “temporal index abnormality/check” column two columns to the right and the “caution or danger/check” column three columns to the right. Details thereof will be described later.

A “one-week trend” column shows the history of intracardiac pressures measured over the past week. The “one-week trend” column will be described later in detail. The “temporal index abnormality/check” column shows the presence or absence of a calculation abnormality recorded in the temporal index value field of the latest record in the patient DB 56 and the presence or absence of checking by a doctor or the like. In a case where there is an abnormality in the temporal index values, hatching is shown in the “temporal index abnormality/check” column. The characters “found” and hatching in the “temporal index abnormality/check” column are examples of second abnormality information indicating that there is a calculation abnormality in the temporal index values.

A “danger or caution/check” column shows results of determination as to the intracardiac pressure values recorded in the determination result field of the latest record in the patient DB 56, and the presence or absence of checking by a doctor or the like. In a case where a determination result is “danger” or “caution”, hatching is shown in the “danger or caution/check” column. The characters “danger” and hatching in the “danger or caution/check” column in a case where a determination result is “danger” are examples of third abnormality information indicating that the intracardiac pressure does not fall within a predetermined range.

Note that the control unit 201 may fill the “temporal index abnormality/check” column and the “danger or caution/check” column with light blue color or the like, instead of hatching. Likewise, the control unit 201 may display the characters “danger” in red and the characters “caution” in orange. With filling with various colors, a patient list screen 71 with high visibility can be achieved.

In the “patient check” column, the presence or absence of checking of the state of the patient recorded in the patient check field in the latest record in the patient DB 56 is recorded. A “notification history” column shows the histories of matters and the results of checking of the patient's state, which have been output to the “caution or danger/check” column in the past. A “measures” column shows the histories of matters with which a doctor or the like has handled for the patient.

The indication mode of the intracardiac pressure shown in the “latest PAP value (mmHg)/measurement date and time” column is now described. The indication for a patient f in the second row from the top, in which the white text is surrounded by a rectangular frame, means that “danger/unchecked” is shown in the “caution or danger/check” column. In a case where a doctor or the like has checked the data of the patient f, the control unit 201 changes the intracardiac pressure to a black character indication surrounded by a rectangular frame as shown in the first row, and shows “danger/checked” in the “caution or danger/check” column. The control unit 201 further updates the data in the determination result field in the patient DB 56. The indication in the second row from the top in the “latest PAP value (mmHg)/measurement date and time” column is an example of the third abnormality information indicating that the intracardiac pressure does not fall within the predetermined range.

Note that the control unit 201 may show the intracardiac pressure of the patient f with white text on a red background, and show the intracardiac pressure of a patient e with numbers in red, for example. Using red color, it is possible to visually and intelligibly indicate a “dangerous” intracardiac pressure.

In the third row from the top, the indication of black characters in which the intracardiac pressure of a patient b is surrounded by a rectangular dashed-line frame indicates that “danger/unchecked” is shown in the “caution or danger/check” column, or that the current intracardiac pressure is within the range of the target value but is predicted to exceed the target value in the future. In a case where a doctor or the like has checked the data of the patient b, the control unit 201 changes the indication of the intracardiac pressure to another mode such as italics, and shows “caution/checked” in the “caution or danger/check” column.

Note that the control unit 201 may show the intracardiac pressure of the patient b with numbers in orange, for example. Using orange color, it is possible to visually and intelligibly indicate that the intracardiac pressure requires “caution”.

The indication in which the intracardiac pressures of a patient c in the fourth row from the top and a patient d at the bottom row are surrounded by a rounded rectangle indicates that “found” is recorded in the calculation abnormality field in the patient DB 56. The indication in which the intracardiac pressure of a patient a in the fifth row from the top is surrounded by a dashed rounded rectangle indicates that “found” is recorded in the calculation abnormality field in the patient DB 56, and that a representative value of the intracardiac pressure cannot be calculated.

Note that the control unit 201 may show the intracardiac pressures of the patient c and the patient d with characters in black on a magenta background, and show the intracardiac pressure of the patient a with characters in black on a fluorescent green background, for example. A combination of a relatively intense color or a clear color and characters in black can visually and intelligibly indicate that, because of an abnormality in the temporal index values, the displayed numerical value should not be believed as it is.

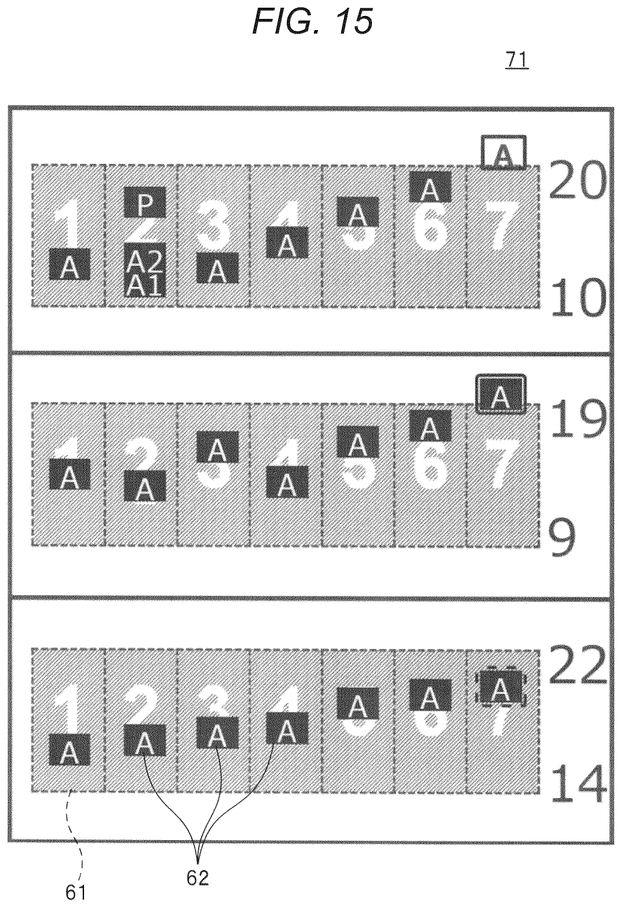

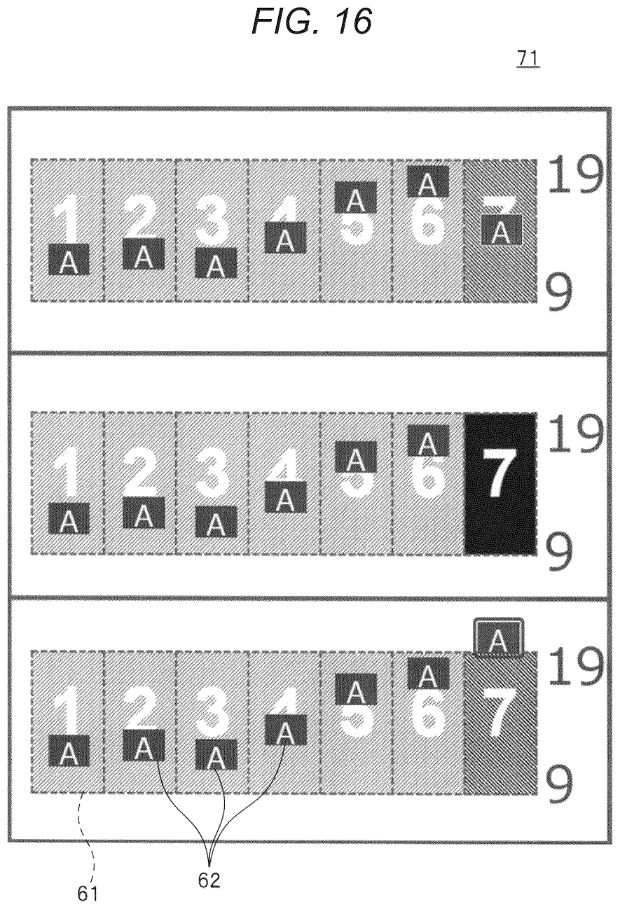

FIG. 15 is an enlarged view of a portion XV in FIG. 14. FIG. 16 is an enlarged view of a portion XVI in FIG. 14. Referring to FIGS. 15 and 16, the indications in the “one-week trend” column are now described.

The control unit 201 displays numbers from “1” to “7” in the “one-week trend” column with white text and hatching. Each of the hatched portions is surrounded by a date frame 61 indicated by a dashed line. The number “1” in white indicates the first day in the display range or six days ago, and the number “7” in white indicates the last day in the display range or the current day. Note that the control unit 201 may show the days of the week, the dates, or the like with white text and hatching, for example.

The numbers at the right end of the “one-week trend” column indicate the intracardiac pressures. For each patient, the upper and lower limits of the target value recorded in the target PAP field of the patient DB 56 are displayed. That is, in the “one-week trend” column, the horizontal axis indicates the dates from six days ago to the current day, and the vertical axis indicates a graph of the intracardiac pressure.

The control unit 201 acquires, from the patient DB 56, the intracardiac pressures from six days ago to the current day, and plots each piece of data with a PAP value index 62 on which a character “A” or “P” is written. “A” indicates that the measurement time is in the morning, and “P” indicates that the measurement time is in the afternoon.

In a case where measurement is performed a plurality of times in one day and results are recorded in the patient DB 56, the control unit 201 plots a plurality of PAP value indexes 62 in the date frame 61 of the one day as shown in a frame with “2” in the top row in FIG. 15. “A1” indicates the first measurement result in the morning, and “A2” indicates the second measurement result in the morning. That is, in a case where the intracardiac pressure is measured a plurality of times in one day, the control unit 201 displays the plurality of intracardiac pressures in the date frame 61 of the one day, using a plurality of PAP value indexes 62. Note that, in a case where there is an extra margin in the horizontal width of the screen, the control unit 201 may shift the PAP value indexes 62 in the horizontal direction in the date frame 61 of the one day, to express the measurement times.

In FIGS. 15 and 16, with respect to any of the patients, the intracardiac pressures till the previous day and the predicted value of intracardiac pressure are within the range of the target value, and “good” is recorded in the determination field of the patient DB 56. The control unit 201 may show the PAP value indexes 62 indicating “good” with white text on a green background, for example. Using green color, it is possible to visually and intelligibly indicate intracardiac pressures that have no problem.

The indication for the patient in the middle row in FIG. 15, in which the white text is surrounded by a rectangular frame, means that “danger/unchecked” is shown in the “caution or danger/check” column, as in the “latest PAP value (mmHg)/measurement date and time” column. Likewise, the indication for the patient in the top row in FIG. 15, in which the intracardiac pressures are surrounded by a rectangular frame, means that “danger/checked” is shown in the “caution or danger/check” column.

If the intracardiac pressure of the patient in the top row in FIG. 15 three days before is out of the range of the target value, and the doctor has not checked the data, the intracardiac pressure is shown in a mode in which white text is surrounded by a square frame in the date frame 61 indicating the date that is three days ago, or the white text “4”, and hatching. In a case where the doctor has already checked the data, the intracardiac pressure is displayed in a mode in which the intracardiac pressure is surrounded by a square frame.

The bottom row in FIG. 15 is an example case where the indication mode of the “latest PAP value (mmHg)/measurement date and time” column is different from the indication mode of the “one-week trend” column. That is, the indication in which white text surrounded by dashed-line frames in the “one-week trend” column, and the indication in which the numbers in black are surrounded by rectangular dashed-line frames in the “latest PAP value (mmHg)/measurement date and time” column both indicate that “danger/unchecked” is shown in the “caution or danger/check” column. By changing the rules for defining the indication mode depending on the size of the numbers indicating the intracardiac pressure, it is possible to achieve screen indications with high visibility.

Note that the control unit 201 may show the intracardiac pressure of the current day in the bottom row in FIG. 15 with white text on an orange background, for example. Using orange color, it is possible to visually and intelligibly indicate that the intracardiac pressure requires “caution”.

The dark hatching inside the date frame 61 of the patient of the current day in the top row and the bottom row in FIG. 16 means that there is an abnormality in the temporal index values. If there is an abnormality in the temporal index values of the patient three days ago in the top row in FIG. 16, dark hatching is shown in the date frame 61 of three days ago or in which the white text “4” and hatching were shown. The dark hatching in the date frame 61 is an example of the second abnormality information indicating that there is a calculation abnormality among the temporal index values.

In a case where the abnormality in the temporal index values is eliminated by the doctor or the like checking and correcting the data as described later, the control unit 201 changes the hatching in the date frame 61 to the mode for a case where there is not an abnormality in the temporal index values.

The black color in the date frame 61 of the current day of the patient in the middle row in FIG. 16 indicates that a representative value of the intracardiac pressure cannot be calculated. As illustrated in FIG. 16, the “one-week trend” column indicates that there is an abnormality in the temporal index values with the hatching and the black color in the date frame 61, and therefore, there is no need to use a rounded square as in the “latest PAP value (mmHg)/measurement date and time” column. Accordingly, in the top row in FIG. 16, PAP value indexes 62 are shown in the same manner as in a case where the intracardiac pressure is good, and, in the bottom row in FIG. 16, PAP value indexes 62 are shown in the same manner as in a case where the intracardiac pressure indicates “danger” and the data is unchecked.

Note that the control unit 201 may show the date frame 61 in the top and bottom rows in FIG. 16 in purple and the date frame 61 in the middle row in FIG. 16 in green, for example. The control unit 201 may show whether there is an abnormality among the temporal index values, whether the intracardiac pressure exceeds the target value, and whether data has been checked by the doctor or the like, in a distinguishable manner and in any appropriate mode. The indication mode under each condition may be set by the user as appropriate.

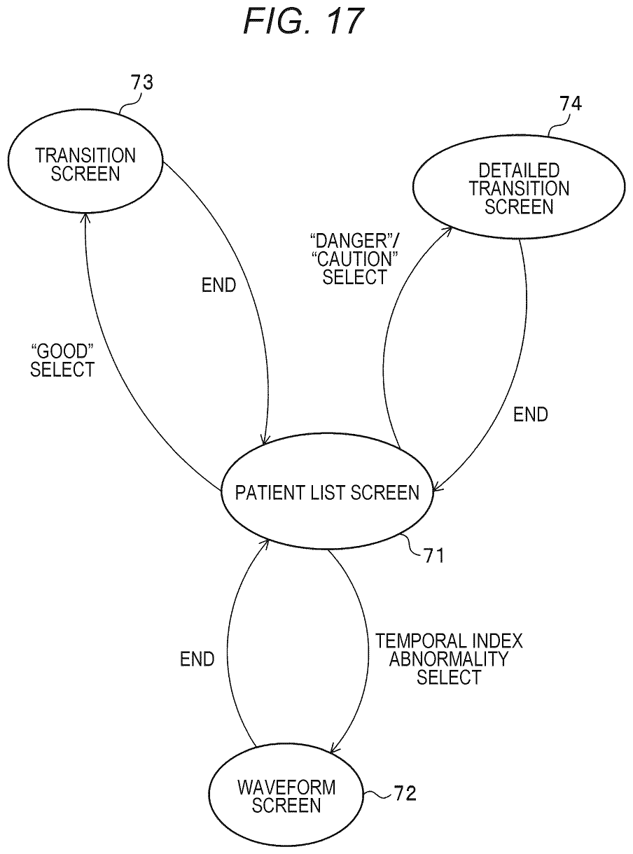

FIG. 17 is a diagram for explaining screen transition. When receiving selection of a portion indicating that there is an abnormality in the temporal index values on the patient list screen 71 shown in the center, the control unit 201 causes the screen display to transition to a waveform screen 72 to be described later. The portions indicating that there is an abnormality among the temporal index values are the inside of a cell in which hatching is shown in the “temporal index abnormality/check” column, and the inside of the date frame 61 in which dark hatching or black color is shown in the “one-week trend” column. In a case where the user issues an instruction to end the display of the waveform screen 72, the control unit 201 returns the screen display to the patient list screen 71.

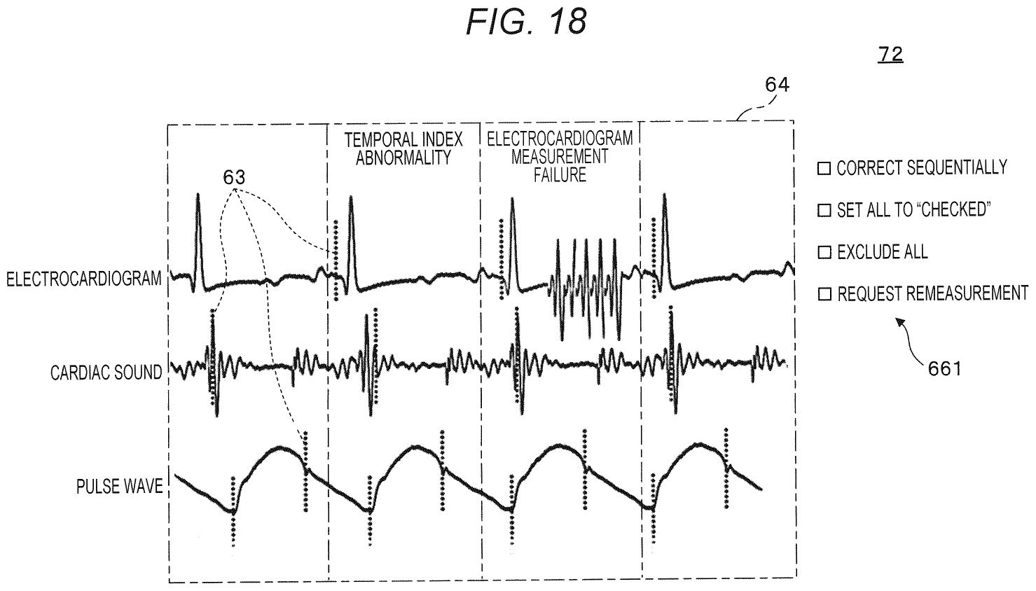

FIGS. 18 to 20 are examples of the waveform screen 72. The waveforms of the electrocardiogram, the cardiac sound, and the pulse wave are shown in this order from the top. The separator for each heartbeat divided in step S702 in the flowchart in FIG. 10 is indicated by a heartbeat frame 64.

The positions of the feature points of the respective waveforms are indicated by feature point indexes 63. A feature point index 63 is an example of information for identifying a feature point determined to have a calculation abnormality in the electrocardiogram data, the cardiac sound data, or the pulse wave data. For a heartbeat having an abnormality among the temporal index values, “temporal index abnormality” is shown in an upper portion of the heartbeat frame 64. A position at which the characters “temporal index abnormality” are shown is an example of the second abnormality information that is output in a case where it is determined that there is a calculation abnormality among the temporal index values. For a heartbeat having an abnormality in the waveforms, an abnormal waveform such as “electrocardiogram measurement failure” and the fact that there is an abnormality are shown in an upper portion of the heartbeat frame 64. The characters “electrocardiogram measurement failure” are an example of first abnormality information indicating that it is determined that there is a measurement abnormality in the electrocardiogram data. The position at which the characters “electrocardiogram measurement failure” are shown is an example of information for identifying the portion determined to have a measurement abnormality in the electrocardiogram data.

Note that the control unit 201 may display the waveform of a portion having a measurement abnormality in a color different from that of the normal portions. For example, the control unit 201 shows the waveform of a normal portion in green, and shows the waveform of a portion having a measurement abnormality in yellow. The color for indicating the waveform of a portion having a measurement abnormality is an example of information for identifying a portion determined to have a measurement abnormality in the electrocardiogram data, the cardiac sound data, or the pulse wave data.

The control unit 201 may show a heartbeat having an abnormality among the temporal index values with a colored background portion. For example, in a case where there is an abnormality in the index PEP and there are no abnormalities in the index PTT and the index STI, the control unit 201 may show the waveforms of the electrocardiogram and the cardiac sound involved in the calculation of the index PEP by coloring the background portion, and show the waveform of the pulse wave in a manner similar to that for the normal portions. The color of the background portion is an example of the first abnormality information.

The control unit 201 may show the waveform of a heartbeat having an abnormality among the temporal index values with a color different from the other portions. For example, in a case where there is an abnormality in the index PEP and there are no abnormalities in the index PTT and the index STI, the control unit 201 shows the waveform of the normal portions in green and the waveform of the portion having the measurement abnormality in blue-green in the waveform of the electrocardiogram that is one of the waveforms involved in the calculation of the index PEP. The colors of the waveforms are an example of the second abnormality information.

Although not shown in the drawings, a horizontal scroll bar is displayed at the upper end or the lower end of the screen, and the user can scroll the waveforms left and right to view.

A first menu field 661 is shown on the right side of the waveform screen 72. In a case where the user's selection of the second item from the top, “set all to ‘checked’”, is received, the control unit 201 changes all “necessary” recorded in the check fields in the analysis DB 57 regarding the measurement data being displayed to “checked”, and changes “unchecked” recorded in the check fields of the waveform field and the temporal index value field in the patient DB 56 to “checked”. On the basis of the changed patient DB 56, the control unit 201 changes the display of the patient list screen 71.

In a case where the user's selection of the third item from the top, “exclude all”, is received, the control unit 201 changes all “N” recorded in the exclusion fields in the analysis DB 57 regarding the measurement data being displayed to “Y”, and changes the calculation abnormality field in the patient DB 56 to “-”. On the basis of the changed patient DB 56, the control unit 201 changes the display of the patient list screen 71.

In a case where the user's selection of “request remeasurement” at the bottom is received, the control unit 201 transmits a message such as “Please remeasure today's data.” to the mobile device 210 being used by the patient corresponding to the measurement data being displayed, via SMS, e-mail, or the like. The selection of “request remeasurement” by the user is an example of a notification instruction to the patient. The message to the mobile device 210 is an example of a notification addressed to the patient.

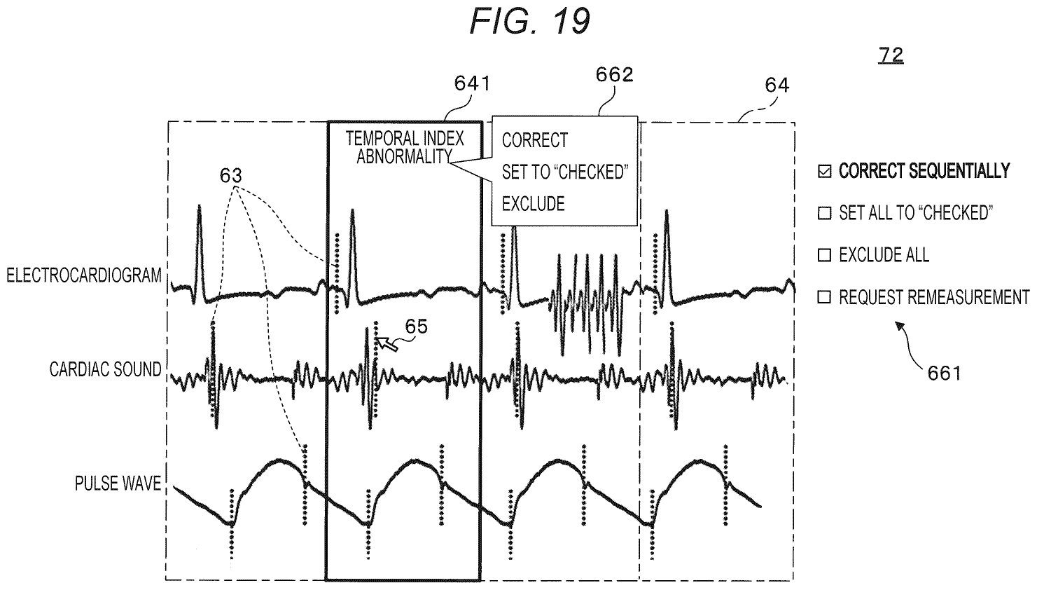

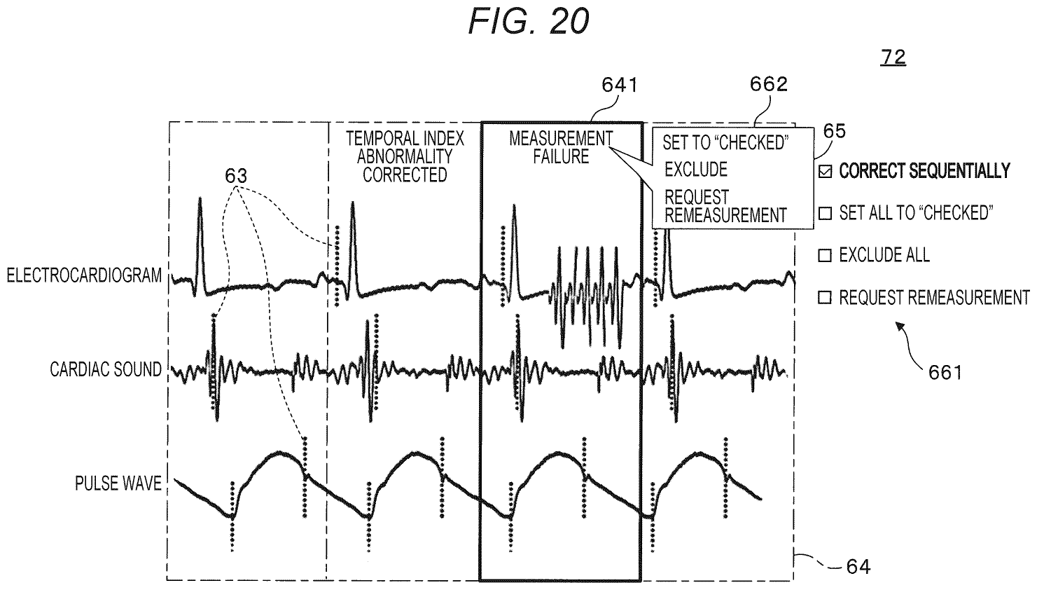

A case where the user's selection of “correct sequentially” at the top is received is now described with reference to FIGS. 19 and 20. The control unit 201 displays a currently-selected heartbeat frame 641 and a second menu field 662 in the first heartbeat in which “necessary” is recorded in the check field in the analysis DB 57.

The characters “temporal index abnormality” shown in FIGS. 18 and 19 are an example of the second abnormality information indicating that it is determined that a calculation abnormality occurs among the temporal index values. The position of the characters “temporal index abnormality” is an example of information for identifying a portion determined to have a calculation abnormality. “Correct”, “set to ‘checked’”, and “exclude” shown in the second menu field 662 in FIG. 19 are an example of options of processing methods for the second abnormality information. When “correct” is selected, for example, the temporal index value (elapsed time) in which a calculation abnormality has occurred is corrected. When “set to ‘checked’” is selected, for example, it is recorded that the doctor has checked the temporal index value (elapsed time) in which a calculation abnormality has occurred. When “exclude” is selected, for example, the temporal index value (elapsed time) in which a calculation abnormality has occurred can be excluded from the representative value calculation.

In FIG. 18, the feature point index 63 corresponding to the feature point S1 is shown as the peak value of the cardiac sound in any heartbeat. However, on the basis of professional judgment by a doctor, a case where the feature point S1 is a peak on the right side in the second heartbeat from the left is described as an example. The doctor selects “correct” from the second menu field 662.

When receiving selection of “correct” from the second menu field 662, the control unit 201 receives a change of the position of the feature point index 63 by the user's operation on a cursor 65. As illustrated in FIG. 19, the doctor moves the feature point index 63 to the peak on the right side of the maximum value.

The control unit 201 may receive a change of the separator position of a heartbeat frame 64. The control unit 201 updates the data in the feature point field in the analysis DB 57 to the position of the feature point index 63 after the moving by the user. The control unit 201 changes “necessary” in the check field to “checked”.

The option “set to ‘checked’” means accepting the current temporal index value. “Accept” may be shown in place of “set to ‘checked’” in the second menu field 662. In a case where the user's selection of “set to ‘checked’” is received, the control unit 201 extracts the field of the corresponding heartbeat from the analysis DB 57, and changes the check field to “checked”. In a case where the user's selection of “exclude” is received, the control unit 201 extracts the field of the corresponding heartbeat from the analysis DB 57, and changes the exclusion field to “Y”.

When receiving the user's selection of “set to ‘checked’” and “exclude”, the control unit 201 does not reflect an operation such as a change of the position of a feature point in the analysis DB 57. In this manner, even in a case where the position of a feature point index 63 has been changed by an erroneous operation or the like, a change in the feature point can be prevented.

As illustrated in FIG. 20, the control unit 201 moves the currently-selected heartbeat frame 641 to the next heartbeat for which “necessary” is recorded in the check field in the analysis DB 57. As illustrated in FIG. 20, in a case where there is an abnormality in the waveforms, the control unit 201 displays the second menu field 662.

The characters “measurement failure” shown in FIG. 20 are an example of the first abnormality information indicating that it is determined that there is a measurement abnormality. The position of the characters “measurement failure” is an example of information for identifying a portion determined to have a measurement abnormality. “Set to ‘checked’”, “exclude”, and “request remeasurement” shown in the second menu field 662 in FIG. 20 are an example of options of processing methods for the first abnormality information.

In a case where the user's selection of “set to ‘checked’” is received, the control unit 201 extracts the field of the corresponding heartbeat from the analysis DB 57, and changes the check field to “checked”. In a case where the user's selection of “exclude” is received, the control unit 201 extracts the field of the corresponding heartbeat from the analysis DB 57, and changes the exclusion field to “Y”.

In a case where the disposed or the fixed state of the measurement apparatus 23 is poor, and an abnormality has occurred in measured waveforms, the user can determine that remeasurement is necessary only by checking the first several waveforms. In a case where the user's selection of “request remeasurement” is received, the control unit 201 transmits a message such as “Please remeasure today's data.” to the mobile device 210 being used by the patient corresponding to the measurement data being displayed, via SMS, e-mail, or the like.

Referring back to FIG. 17, the description is continued. When receiving selection of a portion indicating that the intracardiac pressure is in a preferred state on the patient list screen 71 shown at the center, the control unit 201 causes the screen display to transition to a transition screen 73 to be described later. The portion indicating that the intracardiac pressure is in a preferred state is a cell in which “good” is shown in the “temporal index abnormality/check” column, and a PAP value index 62 in a mode indicating that the intracardiac pressure is in a preferred state in the “one-week trend” column. In a case where the user issues an instruction to end the display of the transition screen 73, the control unit 201 returns the screen display to the patient list screen 71.

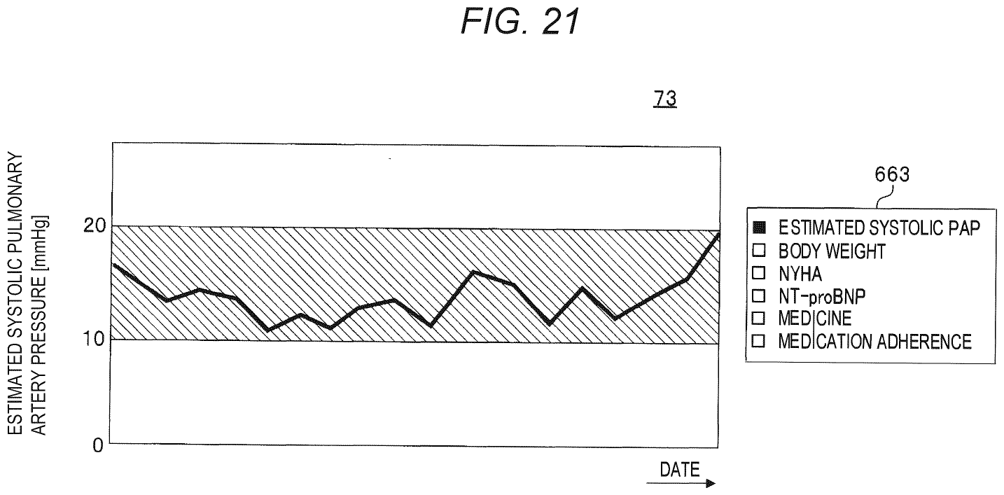

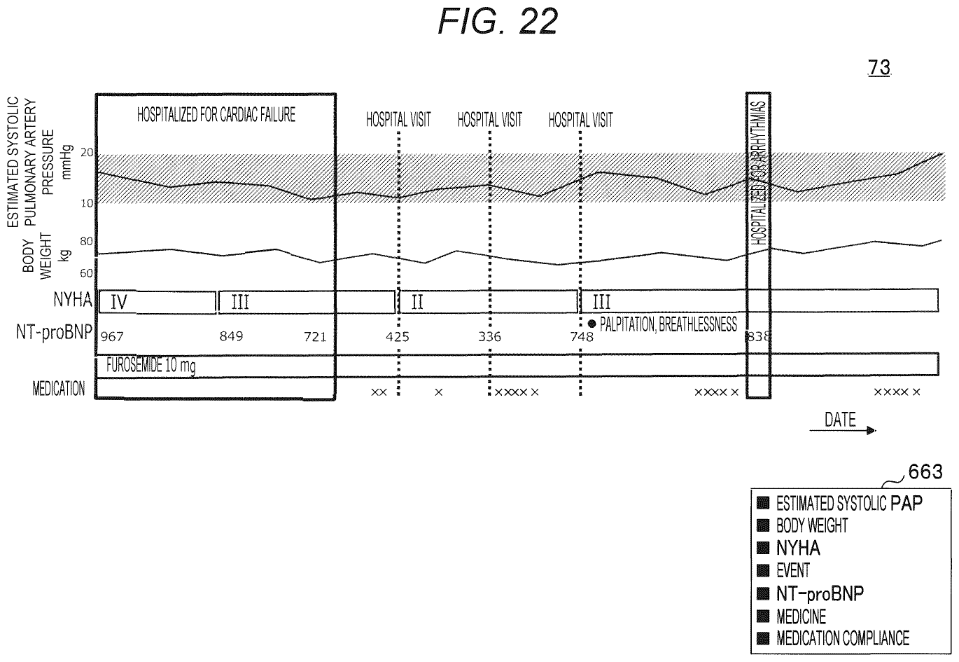

FIGS. 21 and 22 are examples of the transition screen 73. A third menu field 663 is shown at a lower portion of the screen. FIG. 21 illustrates an example of the screen in a case where only “estimated systolic PAP” is selected in the third menu field 663. The past systolic pulmonary artery pressure recorded in the systolic PAP field in the patient DB 56 is indicated with a line graph. The horizontal axis of the graph indicates date, and the vertical axis of the graph indicates systolic pulmonary artery pressure. Hatching indicates the range of target values for systolic pulmonary artery pressure.

FIG. 22 illustrates an example case where “body weight”, “NYHA (New York Heart Association)”, “event”, “NT-proBNP (N-Terminal pro Brain Natriuretic Peptide)”, “medicine”, and “medication compliance” are selected in addition to “estimated systolic PAP” in the third menu field 663.

Under the graph of systolic pulmonary artery pressure similar to that in FIG. 21, a graph showing change in body weight, NYHA classification, events such as symptoms reported by the patient, the value of NT-proBNP, and the name and amount of the medicine prescribed to the patient are shown. Under the name of the medicine, medication compliance is shown with “x”s indicating the date when the medicine was forgotten to be taken. The hospital stay period is indicated by a rectangular frame, and the reason for the hospital stay is written inside the frame. Day visits to the hospital are indicated by dotted lines. The value of NT-proBNP is an example of past examination data. The name and amount of medicine are an example of the history of medicine prescribed to the patient in the past.

The control unit 201 acquires data other than the systolic pulmonary artery pressure from an electronic medical record system (not shown), and displays the screen shown in FIG. 21. The doctor or the like can more accurately grasp the condition of the patient by compiling various kinds of information regarding the patient and the transition of the intracardiac pressure.

Referring back to FIG. 17, the description is continued. In a case where selection of a portion indicating that the intracardiac pressure is not in a preferred state is received on the patient list screen 71 shown at the center, the control unit 201 causes the screen display to transition to a detailed transition screen 74 to be described later. The portion indicating that the intracardiac pressure is not in a preferred state is a cell in which “danger” or “caution” is shown in the “temporal index abnormality/check” column, and a PAP value index 62 in a mode indicating that the intracardiac pressure in the “one-week trend” column is in a state that is dangerous or requires caution. In a case where the user issues an instruction to end the display of the detailed transition screen 74, the control unit 201 returns the screen display to the patient list screen 71.

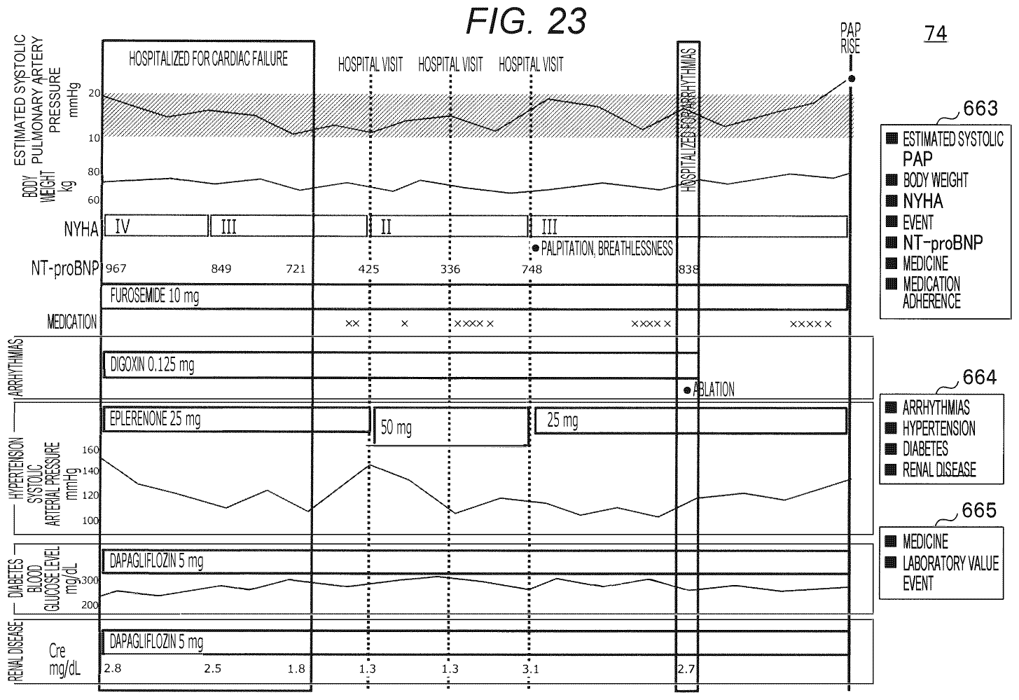

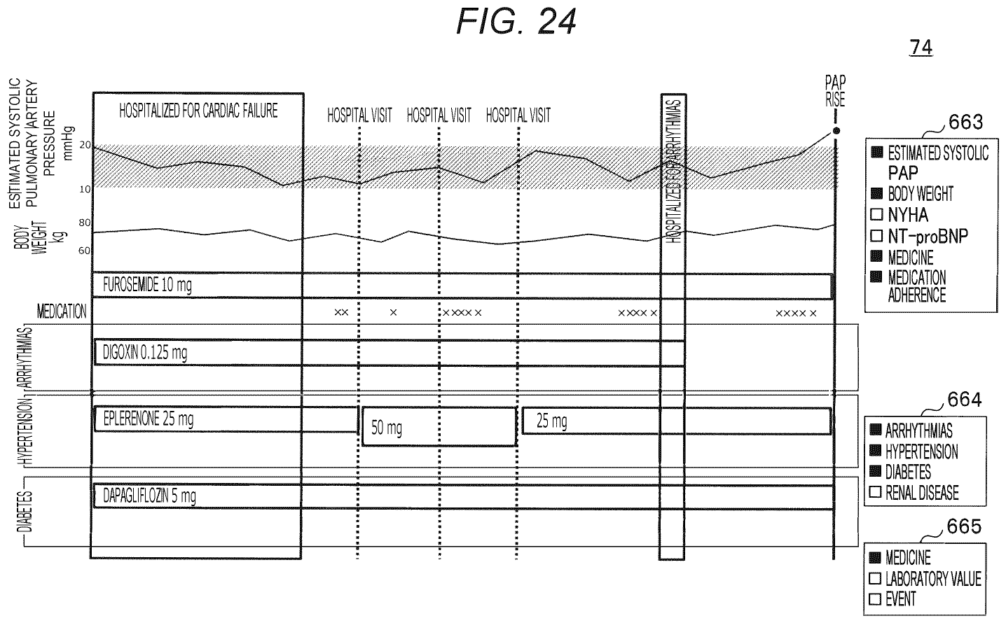

FIGS. 23 and 24 are examples of the detailed transition screen 74. In addition to the third menu field 663, a fourth menu field 664 and a fifth menu field 665 are shown at the right side on the screen. In the fourth menu field 664, options of names for diseases are shown. In the fifth menu field 665, options of indication items related to the disease selected in the fourth menu field 664 are shown.

The third menu field 663 in FIGS. 23 and 24 is different from the third menu field 663 in FIG. 22 in that the lowest item is “medication adherence” as in FIG. 21. In FIG. 23, “estimated systolic PAP”, “body weight”, “NYHA (New York Heart Association)”, “event”, “NT-proBNP”, “medicine”, and “medication adherence” are selected in the third menu field 663. In the fourth menu field 664, the four disease names of “arrhythmia”, “hypertension”, “diabetes”, and “renal disease” are selected. In the fifth menu field 665, “medicine”, “laboratory value”, and “event” are selected.

As in FIG. 22, a graph of the systolic pulmonary artery pressure, a graph showing change in body weight, NYHA classification, events such as symptoms reported by the patient, the value of NT-proBNP, the name of the medicine prescribed to the patient, and medication adherence are shown. The amount of the medicine is also shown on the right side of the name of the medicine. The medication adherence is shown with “x”s indicating the date when the medicine was forgotten to be taken, as in the medication compliance in FIG. 22.

Under the medication adherence, the name of the medicine prescribed for treatment of arrhythmia, an event related to arrhythmia, the name of the medicine prescribed for treatment of hypertension, a graph of systolic blood pressure, which is a laboratory value related to hypertension, the name of the medicine prescribed for treatment of diabetes, a graph of blood glucose level, which is a laboratory value related to diabetes, and the name of the medicine prescribed for treatment of renal disease, and a graph of Cre (creatinine), which is a laboratory value related to renal disease, are shown.

The graph of systolic blood pressure is an example of the past examination data regarding hypertension. The graph of blood glucose level is an example of the past examination data regarding diabetes. The graph of Cre is an example of the past examination data regarding renal disease.

The name of each medicine is written inside a band-like frame. As shown in the medicine for hypertension, for example, the prescribed amount of each medicine is expressed by the thickness of the frame. The hospital stay period is indicated by a rectangular frame, and the characters “hospitalized for cardiac failure” are written as the reason for the hospital stay inside the frame. Day visits to the hospital are indicated by dotted lines.

The control unit 201 acquires data other than the systolic pulmonary artery pressure from an electronic medical record system not shown in the drawing, and displays the screen illustrated in FIG. 23. The control unit 201 displays a comment “PAP rise” to indicate that the systolic pulmonary artery pressure has increased and exceeds the range of the target value in the latest data.

The doctor or the like can more accurately grasp the condition of the patient by compiling the state of treatment regarding the patient and the transition of the intracardiac pressure.

In FIG. 24, “estimated systolic PAP”, “body weight”, “medicine”, and “medication adherence” are selected in the third menu field 663. In the fourth menu field 664, the three disease names of “arrhythmia”, “hypertension”, and “diabetes” are selected. In the fifth menu field 665, “medicine” is selected.

In accordance with the items selected in the third to fifth menu fields 663 to 665, a graph of systolic pulmonary artery pressure, a graph showing change in body weight, the name and medication adherence of the medicine prescribed for the patient, the name of the medicine prescribed for treatment of arrhythmia, the name of the medicine prescribed for treatment of hypertension, a graph of systolic blood pressure as the laboratory value related to hypertension, the name of the medicine prescribed for treatment of diabetes, and the name of the medicine prescribed for treatment of renal disease are shown in this order from the top. The amount of the medicine is also shown on the right side of the name of each medicine.

The doctor or the like can quickly grasp necessary information by narrowing down the display to the items to be focused on.

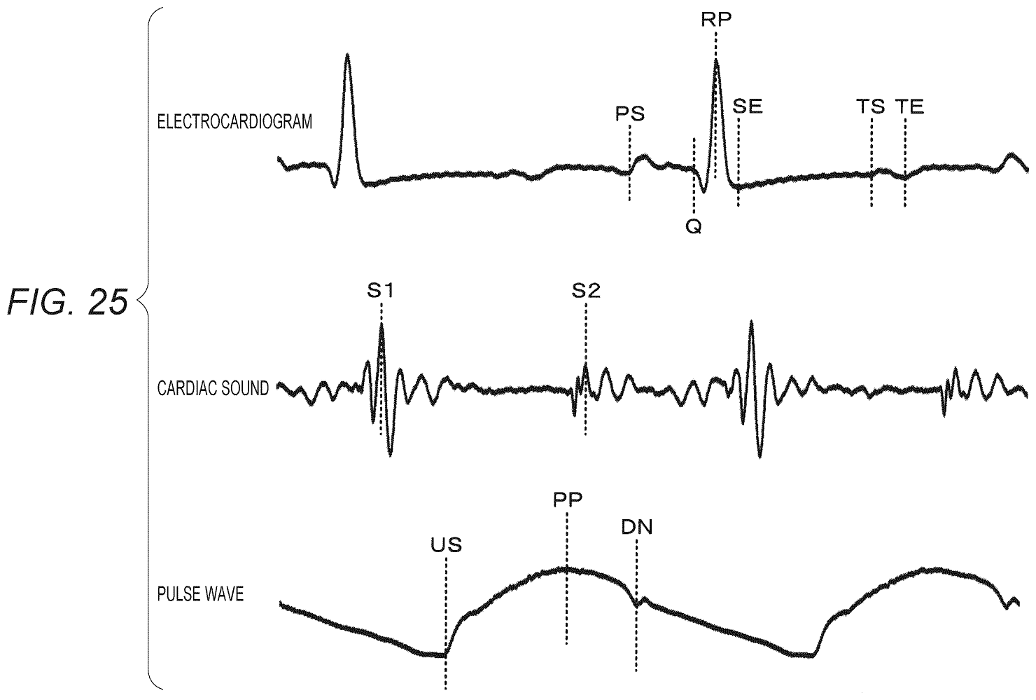

The feature points are not limited to the four described with reference to FIG. 4. FIG. 25 is an explanatory diagram for explaining another example of feature points. A feature point PS indicates a start position of the P wave. A feature point RP indicates a peak position of the R wave. A feature point SE indicates an end position of the S wave. A feature point TS indicates a start position of the T wave. A feature point TE indicates an end position of the T wave. A feature point S2 indicates a diastolic cardiac sound (II sound, S2). A feature point PP indicates a peak position of the pulse wave. Since any of the feature points is commonly used in the field of cardiovascular medicine, a detailed definition thereof is not explained herein.

For example, an elapsed time between any two feature points selected from among these feature points, such as an elapsed time between the feature point PP and the feature point S2, can be used as a temporal index value. Note that the feature points shown in FIG. 25 are an example, and the embodiment is not limited to these feature points. By selecting two feature points, it is possible to define a temporal index value.

According to the present embodiment, it is possible to provide the monitoring system 10 that estimates intracardiac pressure on the basis of the electrocardiogram, the cardiac sound, and the pulse wave that are less invasive during measurement. As the patient can measure data every day at home or the like, it is possible to provide the monitoring system 10 that can detect signs of worsening cardiac failure at an early stage while the patient lives a normal daily life. Furthermore, as the patient or a medical care professional can measure data every day at a medical institution, a nursing home, or the like, it is possible to provide the monitoring system 10 that can detect signs of worsening cardiac failure of the patient at an early stage. Thus, it is possible to provide the monitoring system 10 that reduces the anxiety of a patient about a recurrence of cardiac failure and enhances QOL of the patient.

According to the present embodiment, a representative value is calculated after intracardiac pressure is estimated on the basis of each of several tens of heartbeats measured at one time, and thus, it is possible to provide the monitoring system 10 that derives intracardiac pressure less affected by physiological fluctuations.

According to the present embodiment, it is possible to provide the monitoring system 10 in which the risk of erroneous determination is lowered by calculating intracardiac pressure again after the doctor or the like corrects the position of a feature point on the basis of the technical knowledge as described with reference to FIG. 19.

According to the present embodiment, it is possible to provide the monitoring system 10 that avoids the influence of a measurement error due to accidental noise or the like, by excluding a heartbeat showing a measurement abnormality from the target for estimating the intracardiac pressure.

According to the present embodiment, it is possible to provide the monitoring system 10 with which a doctor or the like can check data of a plurality of patients on one screen, and can check detailed information regarding each of the patients as necessary. According to the present embodiment, it is possible to provide the monitoring system 10 that can request a patient to perform remeasurement in a case where a doctor or the like determines that remeasurement is necessary.

According to the present embodiment, it is possible to provide the monitoring system 10 that displays the fact of being in “danger” to draw attention in a case where an estimated intracardiac pressure exceeds the range of the target value set by the attending doctor. It is also possible to provide the monitoring system 10 that displays “caution” to draw attention in a case where a predicted future intracardiac pressure exceeds the range of the target value set by the attending doctor.

According to the present embodiment, it is possible to provide the monitoring system 10 that displays the treatment states of various diseases and the history of the intracardiac pressure side by side.

### Second Embodiment

The second embodiment relates to a method of creating a first model 51 through machine learning. Explanation of the same portions as those of the first embodiment is not made herein.

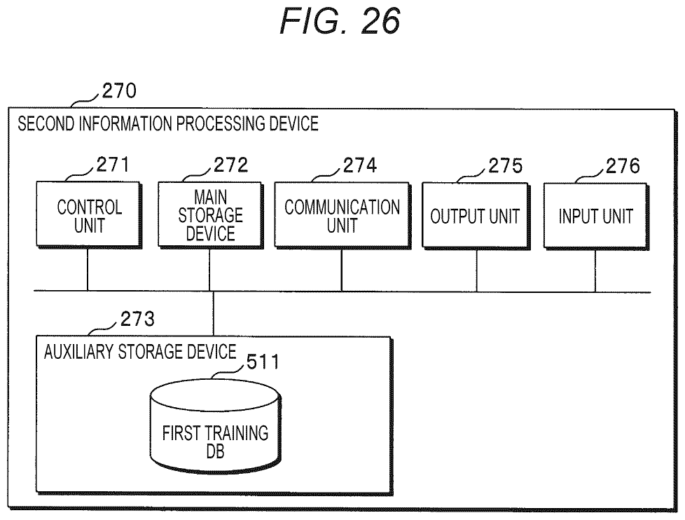

FIG. 26 is a diagram for explaining a configuration of a second information processing device 270. The second information processing device 270 includes a control unit 271, a main storage device 272, an auxiliary storage device 273, a communication unit 274, an output unit 275, an input unit 276, and a bus.

The control unit 271 is an arithmetic control device that executes a program according to the present embodiment. As the control unit 271, one or more CPUs or GPUs, a multi-core CPU, or the like is used. Through the bus, the control unit 271 is connected to each of the hardware components constituting the second information processing device 270.

The main storage device 272 is a storage device such as an SRAM, a DRAM, or a flash memory. The main storage device 272 temporarily stores information necessary during the process to be performed by the control unit 271, and the program being executed by the control unit 271.

The auxiliary storage device 273 is a storage device such as an SRAM, a flash memory, a hard disk, or a magnetic tape. The auxiliary storage device 273 stores a first training DB 511, the program to be executed by the control unit 271, and various kinds of data necessary for executing the program. The first training DB 511 may be stored in an external large-capacity storage device connected to the second information processing device 270.

The communication unit 274 is a network interface circuit that conducts communication between the second information processing device 270 and a network. The output unit 275 is an LCD panel or an organic EL panel, for example. The input unit 276 is a keyboard, a mouse, a voice input microphone, a gesture input sensor, or the like.

The second information processing device 270 is a personal computer, a tablet, a large computing machine, a virtual machine that runs in a large computing machine, or a quantum computer. The second information processing device 270 may be formed with a plurality of personal computers that perform distributed processing, or hardware such as a large computing machine. The second information processing device 270 may be formed with a cloud computing system. The second information processing device 270 may be formed with a plurality of personal computers that operate in conjunction with one another, or hardware such as a large computing machine.

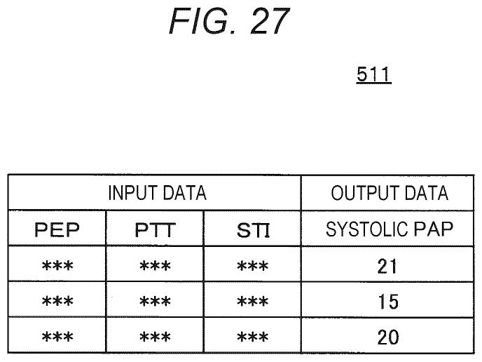

FIG. 27 is a diagram for explaining the record layout in the first training DB 511. The first training DB 511 has an input data field and an output data field. The input data field includes a PEP field, a PTT field, and an STI field. The output data field includes a systolic PAP field. In FIG. 27, each “***” represents a numerical value.

In the respective subfields of the input data field, an index PEP, an index PTT, and an index STI actually measured with a measurement apparatus 23 are recorded. In the systolic PAP field, the systolic pulmonary artery pressure actually measured for the same patient with a Swan-Ganz catheter or the like is recorded. Measurement with the measurement apparatus 23 and measurement with the Swan-Ganz catheter are simultaneously performed, and data measured for the same heartbeat is recorded in one record. The first training DB 511 has one record for one heartbeat.

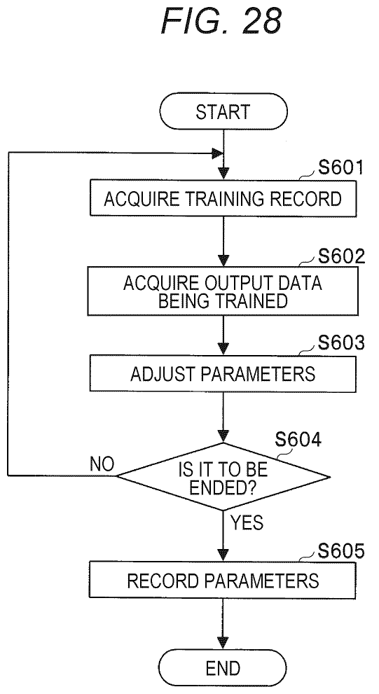

FIG. 28 is a flowchart of processing performed according to a program.

Referring to FIG. 28, the flow of processing according to a program for generating the first model 51 using the first training DB 511 is described. Prior to execution of the program illustrated in FIG. 28, an untrained classification model having a CNN structure or the like is prepared, for example.

The control unit 271 acquires a training record from the first training DB 511 (step S601). The control unit 271 inputs the input data included in the acquired training record to the trained model that is being trained, and acquires output data (step S602). In the description below, the data to be output from the trained model that is being trained will be referred to as the output data being trained.

The control unit 271 adjusts the parameters of the trained model that is being trained, using a technique such as backpropagation, so as to reduce the difference between the input data acquired in step S601 and the output data being trained (step S603).

The control unit 271 determines whether to end the parameter adjustment (step S604). For example, in a case where training is repeated a predetermined number of times defined by a hyperparameter, the control unit 271 determines to end the processing. The control unit 271 may acquire test data from the first training DB 511, input the test data to the trained model that is being trained, and determine to end the processing when an output with a predetermined accuracy is obtained.

If the control unit 271 determines not to end the processing (NO in step S604), the control unit 271 returns to step S601. If the control unit 271 determines to end the processing (YES in step S604), the control unit 271 records the adjusted parameters in the auxiliary storage device 273 (step S605). After that, the control unit 271 ends the processing. Thus, the generation of the first model 51 is completed. The generated first model 51 is transmitted to the information processing device 200 via the network, and is stored into the auxiliary storage device 203.

The first model 51 may be generated, with actual measurement data of a plurality of patients used as training data. The first model 51 may be customized exclusively for one patient by performing additional training with actual measurement data of the one patient on a trained model generated beforehand with actual measurement data of a plurality of patients. Through the additional learning, the first model 51 that can accurately estimate intracardiac pressure for a specific patient can be generated.

### Third Embodiment

The third embodiment relates to a mode in which a monitoring system 10 is implemented by causing a computer 90 and a program 97 to operate in combination. Explanation of the same portions as those of the first embodiment is not made herein.

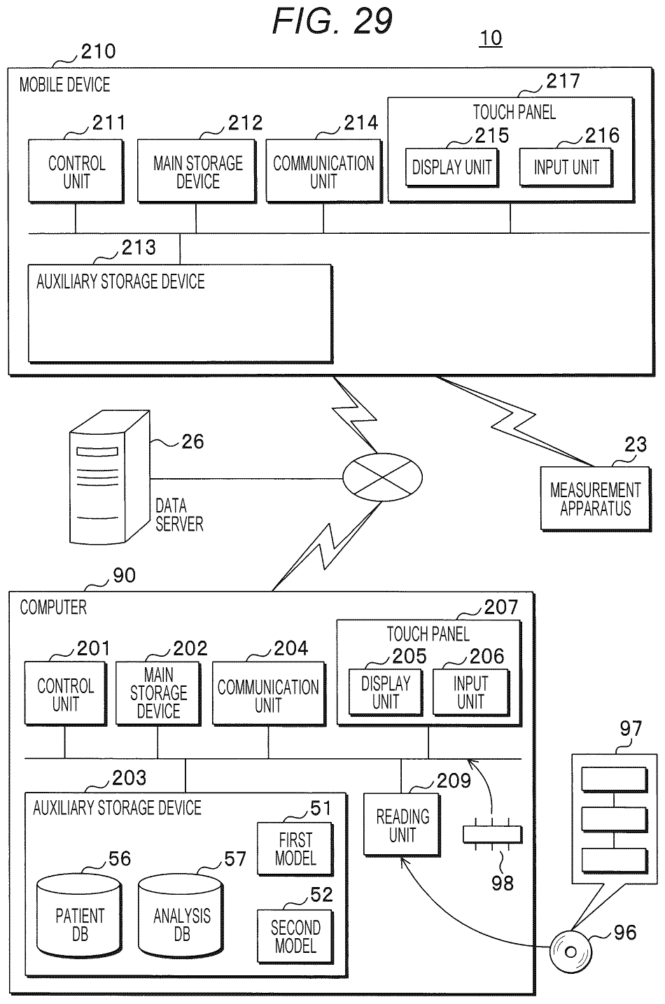

FIG. 29 is a diagram for explaining a configuration of the monitoring system 10 according to the third embodiment. The computer 90 includes a reading unit 209, in addition to the control unit 201, the main storage device 202, the auxiliary storage device 203, the communication unit 204, the touch panel 207, and the bus, which have been described above.

A program 97 is recorded in a portable recording medium 96. The control unit 201 reads the program 97 through the reading unit 209, and stores the program 97 into the auxiliary storage device 203. Alternatively, the control unit 201 may read the program 97 stored in a semiconductor memory 98 such as a flash memory mounted in the computer 90. Further, the control unit 201 may download the program 97 from another server computer (not shown) connected through the communication unit 204 and a network (not shown), and store the program 97 into the auxiliary storage device 203.

The program 97 is installed as a control program for the computer 90, is loaded into the main storage device 202, and is then executed. In this manner, the information processing device 200 described in the first embodiment is implemented.

The computer program can be loaded so as to be executed in a single computer or in a plurality of computers that are disposed at one site or are distributed across a plurality of sites, and are interconnected by a communication network.

The technical features (components) described in the respective embodiments can be combined with each other, and new technical features can be formed by the combinations.

It should be understood that the embodiments disclosed herein are examples in all respects and are not restrictive. The scope of the present invention is indicated not by the above signification but by the claims, and is intended to include all changes within the signification and scope equivalent to the claims.

Some or all of the independent claims and their dependent claims described in the claims can be combined, regardless of their dependent relationships. Furthermore, although a format (multiple dependent claim format) in which a claim dependent on two or more other claims is described is used in the claims, the claim form is not limited to this. The claims may be described in a format (multi-multi claim) in which a multiple dependent claim is dependent on at least one multiple dependent claim.

### Supplementary Note 1

A program for causing a computer to perform a process including:
acquiring electrocardiogram data, cardiac sound data, and pulse wave data;
extracting a feature point of each set of data of the electrocardiogram data, the cardiac sound data, and the pulse wave data;
outputting second abnormality information regarding a calculation abnormality in a temporal index value that is an elapsed time between the feature points, the elapsed time being calculated on the basis of at least two feature points among the feature point of the electrocardiogram data, the feature point of the cardiac sound data, and the feature point of the pulse wave data; and
outputting an option for a processing method for the calculation abnormality in the temporal index value.

### Supplementary Note 2

The program according to supplementary note 1, in which
the temporal index value is an elapsed time between two of the feature points, the elapsed time being calculated on the basis of the feature point of the electrocardiogram data and the feature point of the cardiac sound data, the feature point of the electrocardiogram data and the feature point of the pulse wave data, and the feature point of the cardiac sound data and the feature point of the pulse wave data.

### Supplementary Note 3

The program according to supplementary note 1 or 2, in which
the option is one of the three options of correcting the calculation abnormality in the temporal index value, excluding the calculation abnormality in the temporal index value, and having the calculation abnormality in the temporal index value checked.

### Supplementary Note 4

The program according to any one of supplementary notes 1 to 3, in which
intracardiac pressure derived on the basis of the temporal index value is output.

### Supplementary Note 5

The program according to supplementary note 4, in which
a history of the intracardiac pressure is output.

### Supplementary Note 6

The program according to supplementary note 5, in which,
when the history includes the intracardiac pressure measured a plurality of times a day, a plurality of the intracardiac pressures is output.

### Supplementary Note 7

The program according to any one of supplementary notes 4 to 6, in which
future intracardiac pressure predicted on the basis of the history of the intracardiac pressure is output.

### Supplementary Note 8

The program according to any one of supplementary notes 4 to 7, in which,
when the intracardiac pressure does not fall within a predetermined range, third abnormality information is output.

### Supplementary Note 9

The program according to supplementary note 8, in which,
when checking of the third abnormality information is received from a user, a mode of outputting the third abnormality information is changed.

### Supplementary Note 10

The program according to any one of supplementary notes 1 to 9, in which the process includes:
determining presence or absence of the measurement abnormality for each heartbeat in the electrocardiogram data;
determining presence or absence of the measurement abnormality for each heartbeat in the cardiac sound data;
determining presence or absence of the measurement abnormality for each heartbeat in the pulse wave data; and
outputting first abnormality information including information for identifying a portion determined to have the measurement abnormality when it is determined that there is the measurement abnormality in the electrocardiogram data, the cardiac sound data, or the pulse wave data.

### Supplementary Note 11

The program according to supplementary note 10, in which
a portion determined to have a measurement abnormality related to the first abnormality information, and a portion determined to have a calculation abnormality related to the second abnormality information are output in different modes.

### Supplementary Note 12

The program according to supplementary note 10 or 11, in which
the process further includes outputting an option for a processing method for the first abnormality information.

### Supplementary Note 13

The program according to supplementary note 12, in which
the option is one of the three options of requesting measurement of new electrocardiogram data, cardiac sound data, and pulse wave data, excluding the measurement abnormality, and having the measurement abnormality checked.

### Supplementary Note 14

The program according to supplementary note 13, in which,
when selection of a processing method for the second abnormality information is received,
   a mode of outputting the second abnormality information is changed.

### Supplementary Note 15

The program according to any one of supplementary notes 1 to 14, in which
the second abnormality information is information in which the feature point used to calculate a temporal index value in which it is determined that the calculation abnormality has occurred is associated with the electrocardiogram data, the cardiac sound data, or the pulse wave data.

### Supplementary Note 16

The program according to any one of supplementary notes 1 to 15, in which,
when selection of correcting the calculation abnormality in the temporal index value is received with respect to the second abnormality information,
a portion determined to have the calculation abnormality in the temporal index value corresponding to the second abnormality information is associated with the electrocardiogram data, the cardiac sound data, or the pulse wave data, and is output.

### Supplementary Note 17

An information processing method implemented by a computer to perform a process of:
acquiring electrocardiogram data, cardiac sound data, and pulse wave data;
extracting a feature point of each set of data of the electrocardiogram data, the cardiac sound data, and the pulse wave data;
outputting second abnormality information regarding a calculation abnormality in a temporal index value that is an elapsed time between the feature points, the elapsed time being calculated on the basis of at least two feature points among the feature point of the electrocardiogram data, the feature point of the cardiac sound data, and the feature point of the pulse wave data; and
outputting an option for a processing method for the calculation abnormality in the temporal index value.

### Supplementary Note 18

An information processing device comprising a control unit, in which
the control unit
   acquires electrocardiogram data, cardiac sound data, and pulse wave data,
   extracts a feature point of each set of data of the electrocardiogram data, the cardiac sound data, and the pulse wave data,
   outputs second abnormality information regarding a calculation abnormality in a temporal index value that is an elapsed time between the feature points, the elapsed time being calculated on the basis of at least two feature points among the feature point of the electrocardiogram data, the feature point of the cardiac sound data, and the feature point of the pulse wave data, and
   outputs an option for a processing method for the calculation abnormality in the temporal index value.

What is claimed is:

1. A system for monitoring a cardiac condition of a patient, comprising:
a display;
an interface circuit connectable to a device configured to store measurement data including electrocardiogram data, cardiac sound data, and pulse wave data of the patient;
a memory that stores a program; and
a processor configured to execute the program to perform the steps of:
acquiring the measurement data from the device through the interface circuit,
determining whether there is a measurement abnormality in the measurement data,
upon determining that there is no measurement abnormality in the measurement data, determining a feature point of each of the electrocardiogram data, the cardiac sound data, and the pulse wave data,
calculating time differences between any two of the feature points, and determining whether there is a calculation abnormality in the time differences,
upon determining that there is no calculation abnormality, determining an intracardiac pressure based on the time differences,
generating a first screen showing information of the patient and the intracardiac pressure when determined, and
controlling the display to display the generated screen.

2. The system according to claim 1, wherein
determining whether there is a measurement abnormality in the measurement data includes:
determining whether there is a measurement abnormality in each heartbeat in the electrocardiogram data,
determining whether there is a measurement abnormality in each heartbeat in the cardiac sound data, and
determining whether there is a measurement abnormality in each heartbeat in the pulse wave data.

3. The system according to claim 2, wherein
the steps further include generating a second screen that shows first abnormality information when there is a measurement abnormality in one of the heartbeats in the electrocardiogram data, the cardiac sound data, and the pulse wave data.

4. The system according to claim 3, wherein
the second screen shows:
the electrocardiogram data, the cardiac sound data, and the pulse wave data,
the first abnormality information that identifies a portion of the electrocardiogram data, the cardiac sound data, or the pulse wave data at which there is a measurement abnormality, and
a plurality of selectable objects each corresponding to an action to be taken in response to the measurement abnormality.

5. The system according to claim 4, wherein
the selectable objects include a first object for requesting that the patient perform a measurement of new electrocardiogram data, cardiac sound data, and pulse wave data, a second object for excluding the measurement abnormality, and a third object for confirming that the measurement abnormality has been checked.

6. The system according to claim 1, wherein
determining an intracardiac pressure includes inputting the time differences into a machine learning model to output the intracardiac pressure.

7. The system according to claim 1, wherein
the steps further include determining whether the intracardiac pressure falls within a predetermined range.

8. The system according to claim 7, wherein
the steps further include generating a third screen showing a history of medicines prescribed for the patient and past examination data of the patient together with the intracardiac pressure when the intracardiac pressure does not fall within the predetermined range.

9. The system according to claim 1, wherein
the steps further include:
predicting future intracardiac pressure based on a history of the intracardiac pressure, and
determining whether the intracardiac pressure falls within a predetermined range.

10. The system according to claim 1, wherein
the steps further include generating a second screen that indicates whether there is a calculation abnormality in the time differences.

11. The system according to claim 10, wherein
the second screen shows information for identifying feature points used to calculate one of the time differences in which there is the calculation abnormality.

12. The system according to claim 10, wherein
the second screen shows a plurality of selectable objects each corresponding to an action to be taken in response to the calculation abnormality.

13. The system according to claim 12, wherein
the selectable objects include a first object for correcting the calculation abnormality, a second object for excluding the calculation abnormality, and a third object for confirming that the calculation abnormality has been checked.

14. The system according to claim 1, wherein
the steps further include, upon receipt of a notification instruction for the patient, controlling the interface circuit to transmit a notification to the device.

15. A method for monitoring a cardiac condition of a patient, the method comprising:
acquiring measurement data including electrocardiogram data, cardiac sound data, and pulse wave data of the patient;
determining whether there is a measurement abnormality in the measurement data;
upon determining that there is no measurement abnormality in the measurement data, determining a feature point of each of the electrocardiogram data, the cardiac sound data, and the pulse wave data;
calculating time differences between any two of the feature points, and determining whether there is a calculation abnormality in the calculated time differences;
upon determining that there is no calculation abnormality, determining an intracardiac pressure based on the time differences;
generating a screen showing information of the patient and the intracardiac pressure when determined; and
displaying the generated screen on a display.

16. An information processing device comprising:
a memory that stores an analysis database (DB) that includes:
a patient ID field for storing a patient ID uniquely assigned to a patient,
a heartbeat number field for storing a serial number assigned to each heartbeat within measurement data, and
an abnormality presence/absence field; and
a processor configured to execute a program stored in the memory to perform the steps including:
acquiring electrocardiogram data, cardiac sound data, and pulse wave data for multiple heartbeats of the patient as data for one measurement,
dividing the electrocardiogram data, the cardiac sound data, and the pulse wave data for each heartbeat,
determining, for each of the divided heartbeats, whether there is a measurement abnormality in each of the electrocardiogram data, the cardiac sound data, and the pulse wave data,
for a heartbeat determined to have a measurement abnormality in the electrocardiogram data, the cardiac sound data, or the pulse wave data, recording that an abnormality is found in a corresponding abnormality presence/absence field,
extracting, for a heartbeat determined to have no measurement abnormality, a feature point for each of the electrocardiogram data, the cardiac sound data, and the pulse wave data,
calculating time differences between two feature points of the electrocardiogram data and the cardiac sound data, two feature points of the electrocardiogram data and the pulse wave data, and two feature points of the cardiac sound data and the pulse wave data, and determining whether there is a calculation abnormality in the calculated time differences,
for a heartbeat determined to have a calculation abnormality, recording that an abnormality is found in a corresponding abnormality presence/absence field,
deriving an intracardiac pressure for each heartbeat based on the time difference corresponding to a heartbeat for which an abnormality is not recorded in the abnormality presence/absence field, and
calculating a representative value of the intracardiac pressure corresponding to the data for one measurement from the derived intracardiac pressure for each heartbeat.

17. The information processing device according to claim 16, wherein
the steps include:
when a number or proportion of heartbeats recorded as having an abnormality in the abnormality presence/absence field is equal to or less than a threshold, determining that the representative value can be calculated, calculating the representative value, and recording the calculated representative value in the analysis DB, and
when the number or proportion of heartbeats recorded as having an abnormality in the abnormality presence/absence field exceeds the threshold, determining that the representative value cannot be calculated, and recording in the analysis DB that the representative value cannot be calculated.

18. The information processing device according to claim 16, further comprising:
a display, wherein
the steps further include:
controlling the display to display the electrocardiogram data, the cardiac sound data, and the pulse wave data as a waveform screen, together with a heartbeat frame indicating a separator position for each divided heartbeat,
for a heartbeat having an abnormality in the time differences, displaying second abnormality information on the waveform screen in association with the corresponding heartbeat frame, the second abnormality information indicating that it is determined that there is a calculation abnormality,
receiving a change instruction from a user on the waveform screen, the change instruction being a change of a position of the feature point or a change of the separator position for a heartbeat having an abnormality,
re-deriving an intracardiac pressure for each heartbeat for which the change instruction has been received, and
re-calculating a representative value of the intracardiac pressure corresponding to the data for one measurement, based on the re-derived intracardiac pressure for each heartbeat.

19. The information processing device according to claim 18, wherein
the analysis DB further includes a feature point field for recording feature points, and
the steps further include:
receiving the change instruction from the user for the position of the feature point, and
updating the data in the feature point field of the analysis DB to the position of the feature point after being moved by the user.

20. The information processing device according to claim 16, further comprising:
a display, wherein
the steps further include:
controlling the display to display the electrocardiogram data, the cardiac sound data, and the pulse wave data as a waveform screen, together with a heartbeat frame indicating a separator position for each divided heartbeat, and
for a heartbeat determined to have a measurement abnormality in the electrocardiogram data, the cardiac sound data, or the pulse wave data, displaying first abnormality information on the waveform screen in association with the corresponding heartbeat frame, the first abnormality information indicating that it is determined that there is the measurement abnormality.
